SEQUENCE LISTING (27 sequences)

SEQ ID NO: 1 - DNA, 618 bp, Homo sapiens
  CDS (1)..(615)
atgtcgcgcggcctccagcttctgctcctgagctgcgcctacagcctggctcccgcgacgccggaggtgaaggtggcttgctccgaagatgtggacttgccctgcaccgccccctgggatccgcaggttccctacacggtctcctgggtcaagttattggagggtggtgaagagaggatggagacaccccaggaagaccacctcaggggacagcactatcatcagaaggggcaaaatggttctttcgacgcccccaatgaaaggccctattccctgaagatccgaaacactaccagctgcaactcggggacatacaggtgcactctgcaggacccggatgggcagagaaacctaagtggcaaggtgatcttgagagtgacaggatgccctgcacagcgtaaagaagagacttttaagaaatacagagcggagattgtcctgctgctggctctggttattttctacttaacactcatcattttcacttgtaagtttgcacggctacagagtatcttcccagatttttctaaagctggcatggaacgagcttttctcccagttacctccccaaataagcatttagggctagtgactcctcacaagacagaactggtatga

SEQ ID NO: 2 - protein, 205 aa, Homo sapiens
MSRGLQLLLLSCAYSLAPATPEVKVACSEDVDLPCTAPWDPQVPYTVSWVKLLEGGEERMETPQEDHLRGQHYHQKGQNGSFDAPNERPYSLKIRNTTSCNSGTYRCTLQDPDGQRNLSGKVILRVTGCPAQRKEETFKKYRAEIVLLLALVIFYLTLIIFTCKFARLQSIFPDFSKAGMERAFLPVTSPNKHLGLVTPHKTELV

SEQ ID NO: 3 - DNA, 2051 bp, Mus musculus
  CDS (14)..(601)
gcgctccagccgcatgtcgcaaggcctccagctcctgtttctaggctgcgcctgcagcctggcacccgcgatggcgatgcgggaggtgacggtggcttgctccgagaccgccgacttgccttgcacagcgccctgggacccgcagctctcctatgcagtgtcctgggccaaggtctccgagagtggcactgagagtgtggagctcccggagagcaagcaaaacagctccttcgaggcccccaggagaagggcctattccctgacgatccaaaacactaccatctgcagctcgggcacctacaggtgtgccctgcaggagctcggagggcagcgcaacttgagcggcaccgtggttctgaaggtgacaggatgccccaaggaagctacagagtcaactttcaggaagtacagggcagaagctgtgttgctcttctctctggttgttttctacctgacactcatcattttcacctgcaaatttgcacgactacaaagcattttcccagatatttctaaacctggtacggaacaagcttttcttccagtcacctccccaagcaaacatttggggccagtgacccttcctaagacagaaacggtatgagtaggatctccactggtttttacaaagccaagggcacatcagatcagtgtgcctgaatgccacccggacaagagaagaatgagctccatcctcagatggcaacctttctttgaagtccttcacctgacagtgggctccacactactccctgacacagggtcttgagcaccatcatatgatcacgaagcatggagtatcaccgcttctctgtggctgtcagcttaatgtttcatgtggctatctggtcaacctcgtgagtgcttttcagtcatctacaagctatggtgagatgcaggtgaagcagggtcatgggaaatttgaacactctgagctggccctgtgacagactcctgaggacagctgtcctctcctacatctgggatacatctctttgaatttgtcctgtttcgttgcaccagcccagatgtctcacatctggcggaaattgacaggccaagctgtgagccagtgggaaatatttagcaaataatttcccagtgcgaaggtcctgctattagtaaggagtattatgtgtacatagaaatgagaggtcagtgaactattccccagcagggccttttcatctggaaaagacatccacaaaagcagcaatacagagggatgccacatttatttttttaatcttcatgtacttgtcaaagaagaatttttcatgttttttcaaagaagtgtgtttctttccttttttaaaatatgaaggtctagttacatagcattgctagctgacaagcagcctgagagaagatggagaatgttcctcaaaatagggacagcaagctagaagcactgtacagtgccctgctgggaagggcagacaatggactgagaaaccagaagtctggccacaagattgtctgtatgattctggacgagtcacttgtggttttcactctctggttagtaaaccagatagtttagtctgggttgaatacaatggatgtgaagttgcttggggaaagctgaatgtagtgaatacattggcaactctactgggctgttaccttgttgatatcctagagttctggagctgagcgaatgcctgtcatatctcagcttgcccatcaatccaaacacaggaggctacaaaaaggacatgagcatggtcttctgtgtgaactcctcctgagaaacgtggagactggctcagcgctttgcgcttgaaggactaatcacaagttcttgaagatatggacctaggggagctattgcgccacgacaggaggaagttctcagatgttgcattgatgtaacattgttgcatttctttaatgagctgggctccttcctcatttgcttcccaaagagattttgtcccactaatggtgtgcccatcacccacactatgaaagtaaaagggatgctgagcagatacagcgtgcttacctctcagccatgactttcatgctattaaaagaatgcatgtgaa

SEQ ID NO: 4 - protein, 196 aa, Mus musculus
MSQGLQLLFLGCACSLAPAMAMREVTVACSETADLPCTAPWDPQLSYAVSWAKVSESGTESVELPESKQNSSFEAPRRRAYSLTIQNTTICSSGTYRCALQELGGQRNLSGTVVLKVTGCPKEATESTFRKYRAEAVLLFSLVVFYLTLIIFTCKFARLQSIFPDISKPGTEQAFLPVTSPSKHLGPVTLPKTETV

SEQ ID NO: 5 - DNA, 981 bp, Homo sapiens
  CDS (1)..(978)
atgtctaatggttatgaagaccacatggccgaagactgcaggggtgacatcgggagaacgaatttgatcgtcaactacctccctcagaacatgacccaggatgagttacgaagcctgttcagcagcattggtgaagttgaatctgcaaaacttattcgggataaagtagcaggacacagcttgggctacggctttgtgaactacgtgaccgcgaaggatgcagagagagcgatcaacacgctgaacggcttgaggctccagtcaaaaaccattaaggtgtcgtatgctcgcccgagctcagaggtgatcaaagacgccaacttgtacatcagcgggctcccgcggaccatgacccagaaggacgtagaagacatgttctctcggtttgggcggatcatcaactcgcgggtcctcgtggatcagactacaggtttgtccagaggggttgcgtttatccggtttgacaaacggtcggaggcagaagaggcaattaccagtttcaatggtcataaacccccaggttcctctgagcccatcgcagtgaagtttgcagccaaccccaaccagaacaaaaacgtggcactcctctcgcagctgtaccactcgccagcgcgacggttcggaggccccgttcaccaccaggcgcagagattcaggttctcccccatgggcgtcgatcacatgagcgggctctctggcgtcaacgtgccaggaaacgcctcctccggctggtgcattttcatctacaacctggggcaggatgccgacgaggggatcctctggcagatgtttgggccgtttggtgccgtcaccaatgtgaaagtgatccgcgacttcaacaccaacaagtgcaaagggtttggctttgtgaccatgacaaactatgaagaagccgcgatggccatagccagcctgaacggctaccgcctgggggacaaaatcttacaggtttccttcaaaaccaacaagtcccacaaataa

SEQ ID NO: 6 - protein, 326 aa, Homo sapiens
MSNGYEDHMAEDCRGDIGRTNLIVNYLPQNMTQDELRSLFSSIGEVESAKLIRDKVAGHSLGYGFVNYVTAKDAERAINTLNGLRLQSKTIKVSYARPSSEVIKDANLYISGLPRTMTQKDVEDMFSRFGRIINSRVLVDQTTGLSRGVAFIRFDKRSEAEEAITSFNGHKPPGSSEPIAVKFAANPNQNKNVALLSQLYHSPARRFGGPVHHQAQRFRFSPMGVDHMSGLSGVNVPGNASSGWCIFIYNLGQDADEGILWQMFGPFGAVTNVKVIRDFNTNKCKGFGFVTMTNYEEAAMAIASLNGYRLGDKILQVSFKTNKSHK

SEQ ID NO: 7 - DNA, 36 bp, Homo sapiens
atttaaaagcttatgtcgcgcggcctccagcttctg

SEQ ID NO: 8 - DNA, 42 bp, Homo sapiens
atttaagaattctcaggtagtgtttcggatcttcagggaata

SEQ ID NO: 9 - DNA, 42 bp, Homo sapiens
atttaaaagcttatgctcaggggacagcactatcatcagaag

SEQ ID NO: 10 - DNA, 44 bp, Homo sapiens
atttaagaattctcaaaaagtctcttctttacgctgtgcagggc

SEQ ID NO: 11 - DNA, 39 bp, Homo sapiens
atttaaaagcttatgagctgcaactcggggacatacagg

SEQ ID NO: 12 - DNA, 42 bp, Homo sapiens
atttaagaattctcataccagttctgtcttgtgaggagtcac

SEQ ID NO: 13 - DNA, 43 bp, Homo sapiens
atttaagaattctcagtagaaaataaccagagccagcagcagg

SEQ ID NO: 14 - DNA, 47 bp, Homo sapiens
atttaaaagcttatgttaacactcatcattttcacttgtaagtttgc

SEQ ID NO: 15 - DNA, 42 bp, Homo sapiens
atttaaggatcctttaagaaatacagagcggagattgtcctg

SEQ ID NO: 16 - DNA, 42 bp, Homo sapiens
atttaacccgggtaccagttctgtcttgtgaggagtcactag

SEQ ID NO: 17 - DNA, 42 bp, Homo sapiens
atttaacccgggtttaagaaatacagagcggagattgtcctg

SEQ ID NO: 18 - DNA, 42 bp, Homo sapiens
atttaaggatcctaccagttctgtcttgtgaggagtcactag

SEQ ID NO: 19 - DNA, 60 bp, Homo sapiens
gatcctttaagaaatacagagcggagattgtcctgctgctggctctggttattttctacc

SEQ ID NO: 20 - DNA, 52 bp, Homo sapiens
gaaattctttatgtctcgcctctaacaggacgacgaccgagaccaataaaag

SEQ ID NO: 21 - DNA, 44 bp, Homo sapiens
atttaaggatccttaacactcatcattttcacttgtaagtttgc

SEQ ID NO: 22 - DNA, 1077 bp, Homo sapiens
  CDS (1)..(1077)
atggaaacacaactgtctaatgggccaacttgcaataacacagccaatggtccaaccaccataaacaacaactgttcgtcaccagttgactctgggaacacagaagacagcaagaccaacttaatagtcaactaccttcctcagaacatgacacaggaggaactaaagagtctctttgggagcattggtgaaatagagtcctgtaagcttgtaagagacaaaataacagggcagagcttgggatatggctttgtgaactacattgaccccaaggatgcagagaaagctatcaacaccctgaatggattgagacttcaaaccaaaacaataaaagtttcctatgctcgcccaagttcagcttctatcagagatgcaaatttatatgtcagcggacttccaaaaacaatgacccagaaggagttggaacagcttttttcacaatatggacgcattattacttctcgtattcttgtcgaccaggtcactggcatatcaaggggtgtagggtttattcgatttgacaagcgaattgaggcagaagaagctatcaaaggcctaaatggccagaaacctcccggtgccacggagccaatcactgtaaagtttgctaataacccaagccaaaaaaccaatcaggccatcctttcccagctgtaccagtctccaaacagaaggtatccaggaccgctagctcagcaggcacagcgttttaggttggacaatctgctcaatatggcttatggagtaaagaggttttctccaatgaccattgacggaatgaccagtttggctggaattaatatccctgggcaccctggaacagggtggtgtatatttgtgtacaacctggctcctgacgcagatgagagtatcctgtggcaaatgtttgggccttttggagctgtcaccaatgtgaaggtcatccgtgactttaacaccaataaatgcaaaggttttggatttgtgactatgacaaactatgatgaggctgccatggcgatacgtagcctcaatggataccgtctgggagacagagtactgcaggtctcctttaagacaaacaaaacgcacaaagcc

SEQ ID NO: 23 - protein, 359 aa, Homo sapiens
METQLSNGPTCNNTANGPTTINNNCSSPVDSGNTEDSKTNLIVNYLPQNMTQEELKSLFGSIGEIESCKLVRDKITGQSLGYGFVNYIDPKDAEKAINTLNGLRLQTKTIKVSYARPSSASIRDANLYVSGLPKTMTQKELEQLFSQYGRIITSRILVDQVTGISRGVGFIRFDKRIEAEEAIKGLNGQKPPGATEPITVKFANNPSQKTNQAILSQLYQSPNRRYPGPLAQQAQRFRLDNLLNMAYGVKRFSPMTIDGMTSLAGINIPGHPGTGWCIFVYNLAPDADESILWQMFGPFGAVTNVKVIRDFNTNKCKGFGFVTMTNYDEAAMAIRSLNGYRLGDRVLQVSFKTNKTHKA

SEQ ID NO: 24 - DNA, 1077 bp, Homo sapiens
  CDS (1)..(1077)
atggtcactcagatactgggggccatggagtctcaggtgggggggggcccggccggcccggccctgcccaacgggccactccttggtacaaatggagccactgacgacagcaagaccaacctcatcgtcaactacctgccccagaacatgacccaggatgagttcaagagtctcttcggcagcattggcgacatcgagtcctgcaagttggttcgggacaagatcacaggcagagaccttggctacgggtttgtgaactatcctgaccccaatgatgcagacaaagccatcaacaccctcaacggcctcaaattacagacgaagaccatcaaggtgtcctatgccagacccagttcagcatccatccgggatgctaacctgtacgtcagcgggctccccaagaccatgagccagaaagagatggagcagctcttctcccagtacggccgcatcatcacgtcccgcatcctggtggaccaggtcacaggtgtctctcggggtgtgggattcatccgctttgacaagaggattgaggccgaagaggctatcaaaggactgaatgggcagaagccgctgggcgcagctgagcccatcacagtcaagttcgcgaacaacccaagtcagaagacggggcaggcgctgctcacccacctctaccagtcatccgcccggcgctacgcaggccccctacaccatcagacccagcgtttccggctggacaatttgctcaacatggcctacggcgtcaagaggttctcgccgatcgccatcgatggtatgagcggcctggcgggcgtgggcctgtcggggggcgcggcgggcggctggtgcatcttcgtgtacaacctgtcaccggaggcagacgagagcgtgctgtggcagctgttcgggccttttggggcagtcaccaacgtcaaggtcatccgtgatttcaccaccaacaagtgcaagggtttcggcttcgtgaccatgaccaactatgacgaggcggccatggccatcgccagcctgaacggctatcgcctggccgagcgcgtgctgcaggtctccttcaagaccagcaaacagcacaaggcg

SEQ ID NO: 25 - protein, 359 aa, Homo sapiens
MVTQILGAMESQVGGGPAGPALPNGPLLGTNGATDDSKTNLIVNYLPQNMTQDEFKSLFGSIGDIESCKLVRDKITGRDLGYGFVNYPDPNDADKAINTLNGLKLQTKTIKVSYARPSSASIRDANLYVSGLPKTMSQKEMEQLFSQYGRIITSRILVDQVTGVSRGVGFIRFDKRIEAEEAIKGLNGQKPLGAAEPITVKFANNPSQKTGQALLTHLYQSSARRYAGPLHHQTQRFRLDNLLNMAYGVKRFSPIAIDGMSGLAGVGLSGGAAGGWCIFVYNLSPEADESVLWQLFGPFGAVTNVKVIRDFTTNKCKGFGFVTMTNYDEAAMAIASLNGYRLAERVLQVSFKTSKQHKA

SEQ ID NO: 26 - DNA, 1140 bp, Homo sapiens
  CDS (1)..(1140)
atggttatgataattagcaccatggagcctcaggtgtcaaatggtccgacatccaatacaagcaatggaccctccagcaacaacagaaactgtccttctcccatgcaaacaggggcaaccacagatgacagcaaaaccaacctcatcgtcaactatttaccccagaatatgacccaagaagaattcaggagtctcttcgggagcattggtgaaatagaatcctgcaaacttgtgagagacaaaattacaggacagagtttagggtatggatttgttaactatattgatccaaaggatgcagagaaagccatcaacactttaaatggactcagactccagaccaaaaccataaaggtctcatatgcccgtccgagctctgcctcaatcagggatgctaacctctatgttagcggccttcccaaaaccatgacccagaaggaactggagcaacttttctcgcaatacggccgtatcatcacctcacgaatcctggttgatcaagtcacaggagtgtccagaggggtgggattcatccgctttgataagaggattgaggcagaagaagccatcaaagggctgaatggccagaagcccagcggtgctacggaaccgattactgtgaagtttgccaacaaccccagccagaagtccagccaggccctgctctcccagctctaccagtcccctaaccggcgctacccaggtccacttcaccaccaggctcagaggttcaggctggacaatttgcttaatatggcctatggcgtaaagagactgatgtctggaccagtccccccttctgcttgttcccccaggttctccccaattaccattgatggaatgacaagccttgtgggaatgaacatccctggtcacacaggaactgggtggtgcatctttgtctacaacctgtcccccgattccgatgagagtgtcctctggcagctctttggcccctttggagcagtgaacaacgtaaaggtgattcgtgacttcaacaccaacaagtgcaagggattcggctttgtcaccatgaccaactatgatgaggcggccatggccatcgccagcctcaacgggtaccgcctgggagacagagtgttgcaagtttcctttaaaaccaacaaagcccacaagtcc

SEQ ID NO: 27 - protein, 380 aa, Homo sapiens
MVMIISTMEPQVSNGPTSNTSNGPSSNNRNCPSPMQTGATTDDSKTNLIVNYLPQNMTQEEFRSLFGSIGEIESCKLVRDKITGQSLGYGFVNYIDPKDAEKAINTLNGLRLQTKTIKVSYARPSSASIRDANLYVSGLPKTMTQKELEQLFSQYGRIITSRILVDQVTGVSRGVGFIRFDKRIEAEEAIKGLNGQKPSGATEPITVKFANNPSQKSSQALLSQLYQSPNRRYPGPLHHQAQRFRLDNLLNMAYGVKRLMSGPVPPSACSPRFSPITIDGMTSLVGMNIPGHTGTGWCIFVYNLSPDSDESVLWQLFGPFGAVNNVKVIRDFNTNKCKGFGFVTMTNYDEAAMAIASLNGYRLGDRVLQVSFKTNKAHKS

(19) United States
(12) Patent Application Publication
(10) Pub. No.: US 2002/0165186 A1
(43) Pub. Date: Nov. 7, 2002

(54) Compounds that affect CD83 expression, pharmaceutical compositions comprising said compounds and methods for identifying said compounds

(76) Inventors: Joachim Hauber, Langensendelbach (DE); Alexander Thorsten Prechtel, Lauf an der Pegnitz (DE)

Correspondence Address: Leopold Presser, Esq., SCULLY, SCOTT, MURPHY & PRESSER, 400 Garden City Plaza, Garden City, NY, 11530, US

(21) Appl. No.: 10/025,367

(22) Filed: Dec. 19, 2001

(30) Foreign Application Priority Data
Dec. 20, 2000 (GB) 0031145.6

(51) Int. Cl. A61K 48/00; C07H 21/04

(52) U.S. Cl. 514/44; 536/23.2

(57) ABSTRACT

The present invention relates to compounds that specifically block the binding between a member of the HuR family of proteins and a mRNA encoding a member of the CD83 family of proteins and that reduce expression of a member of the CD83 family of proteins in a cell as well as pharmaceutical compositions comprising such compounds and methods for screening and/or identifying compounds that block the binding between a member of the HuR family of proteins and a mRNA encoding a member of the CD83 family of proteins.

### BACKGROUND OF THE INVENTION

The immune system of mammals must possess the capability to react to a very large number of foreign antigens. Lymphocytes constitute a central element of the immune system because they can recognize antigens and effect a specific, adaptive immune response. Lymphocytes can be divided into two general classes of cells, B lymphocytes which are capable of expressing antibodies and T lymphocytes that can be sub-divided into CD4+ helper T cells and CD8+ cytotoxic T cells. Both of these sub-groups of T lymphocytes are capable of recognizing antigens associated with surface proteins known as the major histocompatibility complex (MHC). The recognition of the MHC occurs throughout the T cell receptor (TCR), a protein complex that is anchored in the cytoplasmic membrane of T cells. The CD8+ T cell receptor exclusively mediates interactions between MHC class I antigens and cytotoxic T cells; the CD4+ T cell receptor exclusively mediates interactions between MHC class II antigens and helper T cells.

The triggering of an immune response does not exclusively progress from T cells alone, but rather, through the interaction of T cells with so-called antigen presenting cells (APCs, also known as accessory cells) and their surface markers (for example MHC II).

These accessory cells can be sub-divided into “simple” APCs whose function is to present antigens and “professional” APCs that, aside from presenting antigens, also have an accessory function in stimulating lymphocytes. APCs themselves do not have antigen specificity but serve as “nature's adjuvant” by presenting antigens to T cells. Aside from mononuclear phagocytes, dendritic cells (DC) are members of the APC type. In fact, DCs are the most potent APC known today and they are the only APC that are also able to stimulate naive T cells.

As a result of their different characteristics and function, two types of dendritic cells have been classified to date: follicular dendritic cells (also known as lymphoid-related DCs) that are present in the lymph nodes, spleen and mucosa-associated lymph tissues and interdigitating dendritic cells (also known as myeloid-derived DCs) that are found in the interstitial space of most organs, in T cell rich zones of the lymph nodes and spleen and are distributed throughout the skin where they are known as Langerhans cells.

Immature dendritic cells, i.e. DCs that are not fully capable of stimulating T cells, have the function of taking up antigens and processing them into MHC-peptide complexes. Stimuli such as TNF-alpha (tumor necrosis factor) and CD40L induce the maturation of dendritic cells and lead to a massive de novo synthesis of MHC class I and MHC class II molecules and to a migration of the DC, for example, from the interstitial space of the internal organs through the blood into the lymph nodes of the spleen and liver. Moreover, increased expression of co-stimulator molecules (for example, CD80, CD86) and adhesion molecules (for example, LFA3) occurs during the migration phase into the secondary lymphoid tissues. Mature DC stimulate T lymphocytes upon arrival in the T cell rich regions of the secondary lymphoid tissue by presenting peptide antigens within the context of MHC class I or MHC class II to these T cells.

Depending on the conditions, DCs can stimulate the activation of a variety of T cells which, in turn, can bring about a differential response of the immune system. For example, as mentioned above, DCs that express MHC class I can cause cytotoxic T cells to proliferate and DCs that express MHC class II can interact with helper T cells. In the presence of mature DCs and the IL-12 that they produce, these T cells mature into Th1 cells that produce interferon-gamma. Interferon-gamma and IL-12 serve together to promote the differentiation of T cells into killer cells. In the presence of IL-4, DCs induce T cells to differentiate into Th2 cells which secrete IL-S and IL-4 that in turn activates eosinophils and assist B cells to produce antibodies (Banchereau, J. and Steinman, R. M. (1998) Nature 392: 245-252).

DCs can also induce a so-called mixed leukocyte reaction (MLR) in vitro, a model for allogenic T cell activation and graft rejection.

Mature DC characteristically express, amongst others (e.g. MHC I and II, CD80/86, CD40) the marker molecule CD83 on their cell surface (Zhou, L.-J. and Tedder, T. F. (1995) J. Immunology, vol. 154: 3821-3835). This is one of the best markers for mature DC known today.

CD83, a molecule from the Ig superfamily of proteins, is a single chain, 43 kDa glycoprotein consisting of 205 amino acids with a transmembrane domain and a 39 amino acid cytoplasmic domain and an Ig-like (V-type) extracellular domain that is expressed very strongly on the cell surface of mature DC. The extracellular domain of the CD83 protein differs from the typical Ig-like domain in that it is encoded by at least two exons: one exon only codes for a half of the Ig-like domain, whereas the other exon encodes the membrane spanning domain (see Zhou, L.-J., Schwarting, R., Smith, H. M. and Tedder, T. F. (1992) J. Immunology, vol. 149: 735-742).

The cDNA encoding CD83 contains a 618 bp open reading frame (see Genbank accession number Z11697 and Zhou, L.-J. et al, supra (1995)).

While the precise function of CD83 remains to be determined, it has been demonstrated that inhibition of CD83 cell surface expression on mature DC by interference with nuclear export of CD83 mRNA leads to a clear reduction in the capacity of these cells to stimulate T cells. (Kruse, M. et al. (2000) J. Exp. Med. 191: 1581-1589). Thus, CD83 appears to be required for DC function.

Therefore, pharmacological intervention with regard to CD83 expression may provide an opportunity to interfere with immune system functions involving, for example, the initiation of primary immune responses, autoimmune diseases, allergies and graft rejection.

The regulation of protein expression in a cell, i.e. the availability of mature mRNA to the translation machinery, is decisively influenced by various post-transcriptional RNA processing steps such as polyadenylation, capping, splicing, transport and localization within the cell and the control of mRNA stability. RNA-protein interactions play an essential role in the regulation of post-transcriptional gene expression.

One such protein is protein ELAV (Embryonic Lethal Abnormal Vision), a protein that was first characterized in the fruit fly Drosophila melanogaster and is essential for the development and maintenance of the nervous system. Deletion mutations in the ELAV gene frequently lead to lethal effects in Drosophila as a result of abnormal or missing nerve development (Antic, D. and Keene, J. D. (1997) Am. J. Hum. Genet. 61: 273-278; Keene, J. D., (1999) PNAS (USA) 96: 5-7)).

The Drosophila ELAV protein has been shown to belong to a superfamily of proteins, the so-called RRM (RNA recognition motif) superfamily, that has been highly conserved during evolution. Other members of this superfamily have been identified in human, rat, mouse, chicken and Xenopus, and Zebrafish. This RRM superfamily encompasses four known human proteins (HuA=HuR (Ma, W.-J. et al. (1996) J. Biol. Chem. 271(14): 8144-8151), Genbank accession number U38175; HuB=He1-N1 (King, P. H. et al. (1994) J. Neurosci. 14(4): 1943-1952), Genbank accession number U12431; HuC (as deposited by Manley, T. and Furneaux, H. M. under Genbank accession number L26405) and HuD (Szabo, A. et al. (1991) Cell 67(2): 325-333, Genbank accession number M62843). All Hu proteins typically contain three RNA binding domains (RRM 1-3) and a hinge region between RRM 2 and 3. The hinge region of HuR (=HuA) contains a shuttling sequence that is known as HNS (Hu nucleocytoplasmic shuttling) (Fan, X. C. and Steitz, J. A. (1998) PNAS (USA) 95:15293-15298). This sequence contains a signal for transport in the nucleus, a so-called NLS (nuclear localization signal) as well as a signal for nuclear export, a so-called NES (nuclear export signal).

While HuR has been shown to be ubiquitously expressed in all proliferating cells, HuB, HuC and HuD are expressed in a neuron-specific manner.

Although the exact function of these ELAV or ELAV-like proteins is not known, they are thought to be involved in the regulation of the transport, stability and translation of a group of early response gene (ERG) mRNAs such as those that code for proto-oncoproteins and cytokines. Thus, HuR has been implicated in regulating the transport, stability and translation of mRNAs such as GM-CSF, IL-2, c-myc, c-fos and GLUT1. This regulation has been demonstrated to occur via HuR binding to so-called ARE(s) (AU-rich elements) that are found within these mRNA molecules. AREs are sequence elements that often have the motif AUUUA typically within an AU-rich background, range in size from about 50 to 150 bases, and are present in the 3′ non-translated regions of mRNA molecules where they are thought to act as destabilizing elements in the mRNA molecules in which they occur (see Chen, C.-Y. A. and Shyu, A.-D. (1995) TIBS 20: 465-470). For example, HuR has been shown to bind to a core element of c-fos mRNA of 27 nucleotides that contain the AREs AUUUA, AUUUUA and AUUUUUA (Ma, W.-J. et al. (1996) J. Biol. Chem. 271(14): 8144-8151). It is thought that binding of HuR protein to the ARE(s) of a given mRNA molecule can protect the mRNA molecule from degradation by cellular enzymes.

An object of the present invention is to provide compounds that are capable of inhibiting CD83 expression and/or induction of the T cell stimulatory mode of DC and/or induction of so-called “regulatory cells”.

A further object of the present invention is to provide pharmaceutical compositions comprising compounds that are capable of inhibiting CD83 expression and/or induction of the T cell stimulatory mode of DC and/or induction of so-called “regulatory cells”.

A further object of the present invention is to provide an assay that is useful in screening and/or identifying compounds that are capable of inhibiting CD83 expression and/or induction of the T cell stimulatory mode of DC and/or induction of so-called “regulatory cells” and as such are useful as pharmaceutical agents.

### BRIEF DESCRIPTION OF THE DRAWINGS

FIG. 1: Map of the pcDNA3-CD83 vector.

FIG. 2: Map of the pcDNA3-ELAV (HuR) vector.

FIG. 3: RNA gel mobility shift experiments with CD83 RNA (nucleotides 1 to 618 of SEQ ID NO:1); lane A: CD83 RNA; lane B: CD83 RNA + GST; lane C: CD83 RNA+GST-ELAV (HuR).

FIG. 4: RNA gel mobility shift experiments with CD83 RNA (nucleotides 1 to 618 of SEQ ID NO:1) and different proteins; lane A: CD83 RNA without added protein; lane B: CD83 RNA+GST-M9; lane C: CD83 RNA+GST-L5; lane D: CD83 RNA+GST-ELAV (HuR); lane E: CD83 RNA+GST.

FIG. 5: RNA gel competition mobility shift experiments with CD83 RNA (nucleotides 1 to 618 of SEQ ID NO:1) using increasing amounts of unlabeled CD83 RNA; lane A: labeled CD83 RNA without added unlabeled CD83 RNA; lanes B-G: labeled CD83 RNA with increasing amounts of unlabeled CD83 RNA.

FIG. 6: RNA gel mobility shift experiments with CD83 RNA (nucleotides 1 to 618 of SEQ ID NO:1) and sub-fragments with and without added ELAV (HuR) protein; lane A: CD83 RNA; lane B: CD83 RNA+GST-ELAV (HuR); lane C: CD83 RNA (nucleotides 1 to 294 of SEQ ID NO:1); lane D: CD83 RNA (nucleotides 1 to 294 of SEQ ID NO:1)+GST-ELAV (HuR); lane E: CD83 RNA (nucleotides 202 to 414 of SEQ ID NO:1); lane F: CD83 RNA (nucleotides 202 to 414 of SEQ ID NO:1)+GST-ELAV (HuR); lane G: CD83 RNA (nucleotides 295 to 618 of SEQ ID NO:1); lane H: CD83 RNA (nucleotides 295 to 618 of SEQ ID NO:1)+GST-ELAV (HuR).

FIG. 7: Secondary structure model of a DNA molecule from nucleotides 412 to 618 of SEQ ID NO:1 having an energy value of −41.8 kcal/mol as calculated at 37° C. with the aid of the GCG program MFOLD.

FIG. 8: Secondary structure model of a DNA molecule from nucleotides 412 to 618 of SEQ ID NO:1 having an energy value of −41.7 kcal/mol as calculated at 37° C. with the aid of the GCG program MFOLD.

FIG. 9: Secondary structure model of a DNA molecule from nucleotides 412 to 618 of SEQ ID NO:1 having an energy value of −40.8 kcal/mol as calculated at 37° C. with the aid of the GCG program MFOLD.

FIG. 10: RNA gel mobility shift experiments with sub-fragments of CD83 RNA with and without added ELAV (HuR) protein; lane A: CD83 RNA (nucleotides 295 to 465 of SEQ ID NO:1); lane B: CD83 RNA (nucleotides 295 to 465 of SEQ ID NO:1)+GST-ELAV (HuR); lane C: CD83 RNA (nucleotides 466 to 618 of SEQ ID NO:1); lane D: CD83 RNA (nucleotides 466 to 618 of SEQ ID NO:1)+GST-ELAV (HuR).

FIG. 11: Cloning scheme for the reporter construct pBC12/CMV/CAT.

FIG. 12: CAT activity of the reporter construct pBC12/CMV/CAT without an insert containing CD83 coding sequences in comparison to the reporter construct pBC12/CMV/CAT with a CD83 insert containing nucleotides 412 to 615 of SEQ ID NO:1 as measured in a transient transfection assay using COS cells.

FIG. 13: CAT activity of the reporter construct pBC12/CMV/CAT with a CD83 insert containing nucleotides 412 to 615 of SEQ ID NO:1 as measured in a transient transfection assay using COS cells that were or were not co-transfected with pcDNA-ELAV (HuR).

FIG. 14: CAT activity of the reporter construct pBC12/CMV/CAT with a CD83 insert containing nucleotides 412 to 615 of SEQ ID NO:1 in the sense or antisense orientation as measured in a transient transfection assay using COS cells that were or were not co-transfected with pcDNA-ELAV (HuR) and express ELAV (HuR).

FIG. 15: CAT activity of the reporter construct pBC12/CMV/CAT with a CD83 insert containing nucleotides 412 to 615 of SEQ ID NO:1, nucleotides 412 to 465 of SEQ ID NO:1 or nucleotides 466 to 615 of SEQ ID NO:1 as measured in a transient transfection assay using COS cells that were or were not co-transfected with pcDNA-ELAV (HuR).

FIG. 16: Secondary structure model of a DNA molecule from nucleotides 466 to 615 of SEQ ID NO:1 having an energy value of −29.7 kcal/mol as calculated at 37° C. with the aid of the GCG program MFOLD.

FIG. 17: Secondary structure model of a DNA molecule from nucleotides 466 to 615 of SEQ ID NO:1 having an energy value of −28.4 kcal/mol as calculated at 37° C. with the aid of the program GCG program MFOLD.

### SUMMARY OF THE INVENTION

It has been surprisingly found that HuR has the ability to specifically bind to mRNA molecules encoding the mature DC marker protein CD83. Moreover, it was unexpectedly found that this specific binding of HuR to CD83 mRNA occurs as a result of the interaction between HuR and a cis-active element comprising a portion of the coding region of the CD83 mRNA molecule and that this interaction leads to the increased expression of mRNA molecules comprising this portion of the CD83 mRNA.

Therefore, compounds that are capable of specifically blocking the interaction between a member of the HuR family of proteins and a mRNA encoding a member of the CD83 family of proteins will prevent correct intracellular transport and/or stabilization of said mRNA which, in turn, will lead to decreased expression of the protein encoded by said mRNAs.

The present invention provides such compounds.

Thus, the present invention relates to compounds that specifically block the binding between a member of the HuR family of proteins and a mRNA encoding a member of the CD83 family of proteins and that reduce expression of a member of the CD83 family of proteins in a cell.

The nucleic acid sequence encoding human CD83 protein as well as the amino acid sequence of CD83 are described in Zhou, L. J. et al. (1992) J. Immunol. 149(2): 735−742 (Genbank accession number Z11697) and are provided in SEQ ID NO:1 and SEQ ID NO:2 respectively.

As defined herein, a member of the CD83 family of proteins includes any naturally occurring protein that has at least 70%, preferably 80%, and more preferably 90% or more amino acid identity to the human CD83 as depicted in SEQ ID NO:2, resulting in specific binding of ELAV proteins to the CD83 cis-active RNA sequence of this protein.

Thus, aside from CD83 itself, members of the CD83 family of proteins include the mouse HB15 protein that is encoded by the nucleic acid sequence of SEQ ID NO:3 and is represented by the amino acid sequence provided in SEQ ID NO:4, (Genbank accession number NM_{—}009856 (Berchthold et al).

A preferable member of the CD83 family of proteins for the purpose of the invention is the CD83 protein as shown in SEQ ID NO:2.

The nucleic acid sequence encoding HuR protein as well as the amino acid sequence of HuR are described in Ma, W.-J. et al. (1996) J. Biol. Chem. 271(14): 8144−8151 (GenBank accession number U38175) and are provided in SEQ ID NO:5 and SEQ ID NO:6 respectively.

As defined herein, a member of the HuR family of proteins includes any naturally occurring protein that has 90% or more amino acid identity to HuR. Thus, aside from HuR itself, members of the HuR family of proteins include the proteins encoded by the elrA gene of Xenopus (Good, P. J. (1995) PNAS (USA) 92(10): 4557-4561; Genbank accession number U17596; the mouse me1G protein (Atasoy, U. et al. (1998) J. Cell Science 111: 3145-3156; GenBank accession number U65735)), chicken HuA protein (Wakamatsu, Y. and Weston, J. A. (1997) Development 124(17): 3449-3460; GenBank accession number AF176673); zebrafish HuA protein (Genbank accession number AF184244) and HuG protein (Genbank accession number AF184245).

A preferable member of the HuR family of proteins for the purpose of the invention is the HuR protein as shown in SEQ ID NO:6.

As defined herein, the term “specifically block” is used to indicate that the compounds of the present invention are capable of disrupting the interaction between a member of the HuR family of proteins and a mRNA encoding a member of the CD83 family of proteins at physiological pH and salt concentrations, preferably, at pH concentrations ranging from pH 6.0 to 8.0 and/or at salt concentrations ranging from 50 mM to 250 mM, preferably 125 mM to 175 mM.

A preferred assay for measuring the expression of a member of the CD83 family of proteins in a cell is provided in the examples. Other useful assays for this purpose include fluorescence/indirect immunofluorescence assays, Western analysis, ELISA analysis, immunoprecipitation assays, other assays based on the expression of a reporter gene, such as chloramphenicol acetyltransferase (CAT), luciferase, beta-galactosidase and GFP/BFP, FACS analysis, direct protein gel analysis with staining of the gel and coupled in vitro transcription/translation assays.

A preferred assay for determining the binding between a member of the HuR family of proteins and a mRNA encoding a member of the CD83 family of proteins is provided in the examples. Other useful assays for this purpose include filter binding assays, Biacore interaction analysis (Biacore, Uppsala, Sweden), Scintillation Proximity Assay (Amersham Pharmacia Biotech, Freiburg, Germany), RNAse protection assays, cell-based RNA binding assays (see Blair et al. (1998) RNA 4: 215-225), yeast 3-hybrid assays (for example, RNA-Protein Hybrid Hunter® System (Invitrogen, Groningen, The Netherlands), and reporter gene assays in eukaryotic cells as described above.

The compounds of the present invention cause a disruption in the interaction between a member of the HuR family of proteins and a mRNA encoding a member of the CD83 family of proteins that can be reversible or irreversible and preferably correspond to an inhibition of the binding between a member of the HuR family of proteins and a mRNA encoding a member of the CD83 family of proteins of at least 25%, more preferably at least 50%, still more preferably at least 75% and most preferably at least 90% or greater as measured in one or more of the above assays.

In one embodiment, compounds of the invention comprise nucleic acid molecules that specifically block the binding between a member of the HuR family of proteins and a mRNA encoding a member of the CD83 family of proteins and that reduce expression of a member of the CD83 family of proteins in a cell.

The nucleic acids of the present invention can be in the form of DNA (deoxyribonucleic acid) which contains the bases adenine, thymine, guanine and cytosine or RNA (ribonucleic acid) which contains the bases adenine, uracil, guanine and cytosine or mixtures of the two.

Preferably, these compounds comprise a nucleic acid molecule that contains at least a portion of the coding region of a member of the CD83 family of proteins or a derivative thereof.

When the nucleic acid molecule of the invention is derived from a portion of the coding region of human CD83 protein, the portion of the coding region is preferably from nucleotide 466 to 618 of the sequence in SEQ ID NO:1 or a derivative thereof. Alternatively, the portion of the coding region is from nucleotide 466 to 615 of the sequence in SEQ ID NO:1.

When the nucleic acid molecule of the invention is derived from a portion of the coding region of the mouse HB15 protein, the portion of the coding region is preferably from about nucleotide 14 to 604 of the sequence in SEQ ID NO:3. Alternatively, the portion of the coding region is from nucleotide 14 to 601 of the sequence in SEQ ID NO:3.

Other naturally occuring nucleic acids that can be used for the present invention can be obtained by hybridizing a nucleic acid comprising, for example, all or a portion of the human CD83 coding region or mouse HB15 coding region to various sources of nucleic acids (genomic DNA, cDNA, RNA) from other animals, preferably mammals, or from other tissues of the same organism.

Hybridization refers to the binding between complementary nucleic acid sequences (e.g., sense/antisense). As is known to those skilled in the art, the Tm (melting temperature) refers to the temperature at which the binding between sequences is no longer stable. As used herein, the term “selective hybridization” refers to hybridization under moderately stringent or highly stringent conditions, which can distinguish CD83 related nucleotide sequences from unrelated sequences.

In nucleic acid hybridization reactions, the conditions used in order to achieve a particular level of stringency will vary, depending on the nature of the nucleic acids being hybridized. For example, the length, degree of sequence complementarity, sequence composition (e.g., the GC v. AT content), and type (e.g., RNA v. DNA) of the hybridizing regions can be considered in selecting particular hybridization conditions. An additional consideration is whether one of the nucleic acids is immobilized, for example, on a filter.

In general, the stability of a nucleic acid hybrid decreases as the sodium ion decreases and the temperature of the hybridization reaction increases. An example of moderate stringency hybridization reaction is as follows: 2×SSC/0.1 SDS at about 37° C. or 42° C. (hybridization conditions); 0.5×SSC/0.1% SDS at about room temperature (low stringency wash conditions); 0.5×SSC/0.1 % SDS at about 42° C. (moderate stringency wash conditions). An example of high stringency hybridization conditions is as follows: 2×SSC/0.1% SDS at about room temperature (hybridization conditions); 0.5×SSC/0.1% SDS at about room temperature (low stringency wash conditions); 0.5×SSC/0.l% SDS at about 42° C. (moderate stringency wash conditions); and 0.1×SSC/0.1% SDS at about 65° C. (high stringency conditions).

Typically, the wash conditions are adjusted so as to attain the desired degree of stringency. Thus, hybridization stringency can be determined, for example, by washing at a particular condition, e.g., at low stringency conditions or high stringency conditions, or by using each of the conditions, e.g., for 10-15 minutes each, in the order listed above, repeating any or all of the steps listed. Optimal conditions for selective hybridization will vary depending on the particular hybridization reaction involved, and can be determined empirically.

The compounds of the present invention also include derivatives of each of the nucleic acid molecules according to the invention as mentioned above in which one or more nucleotides has been added, deleted, substituted, inserted or inverted but still specifically block the binding between a member of the HuR family of proteins and a mRNA encoding a member of the CD83 family of proteins and that reduce expression of a member of the CD83 family of proteins in a cell.

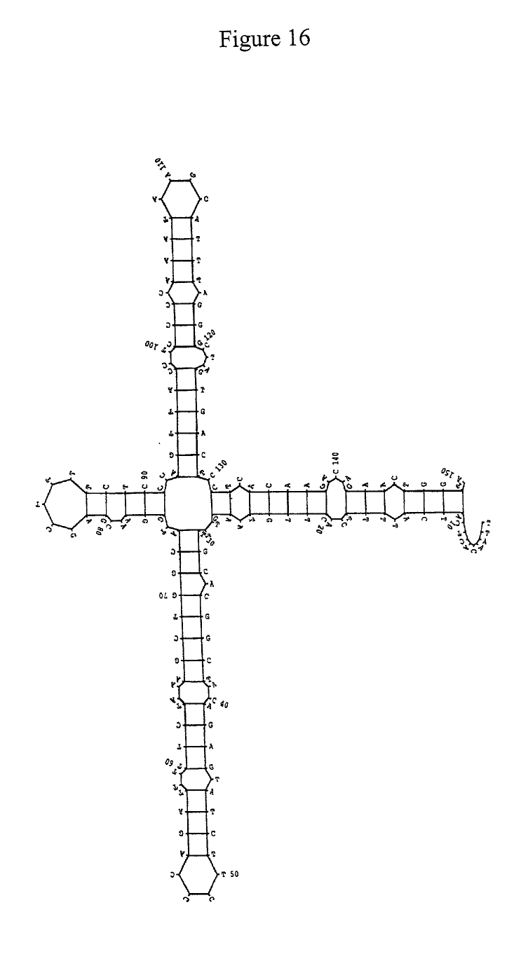

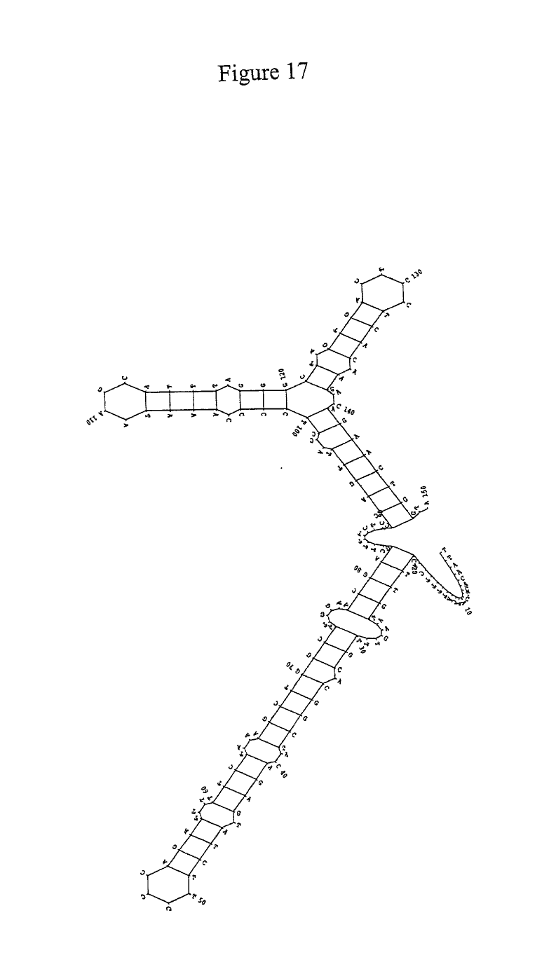

The nucleic acids of the invention can have a secondary structure corresponding to that shown in FIG. 16 comprising a 3-pronged stem-loop structure or FIG. 17 or a similar secondary structure having an energy of −28.4 kcal/mol or less, preferably −29.7 kcal/mol or less when analyzed according to the MFOLD program available from the University of Wisconsin, USA at (http://www.qcq.com) using a folding temperature of 37° C., a maximum size of interior loop of 30 and maximum loopsideness of an interior loop of 30 and nucleotide sequence in the form of a DNA molecule having nucleotides that correspond to the nucleotide sequence from 466 to 615 of SEQ ID NO:1.

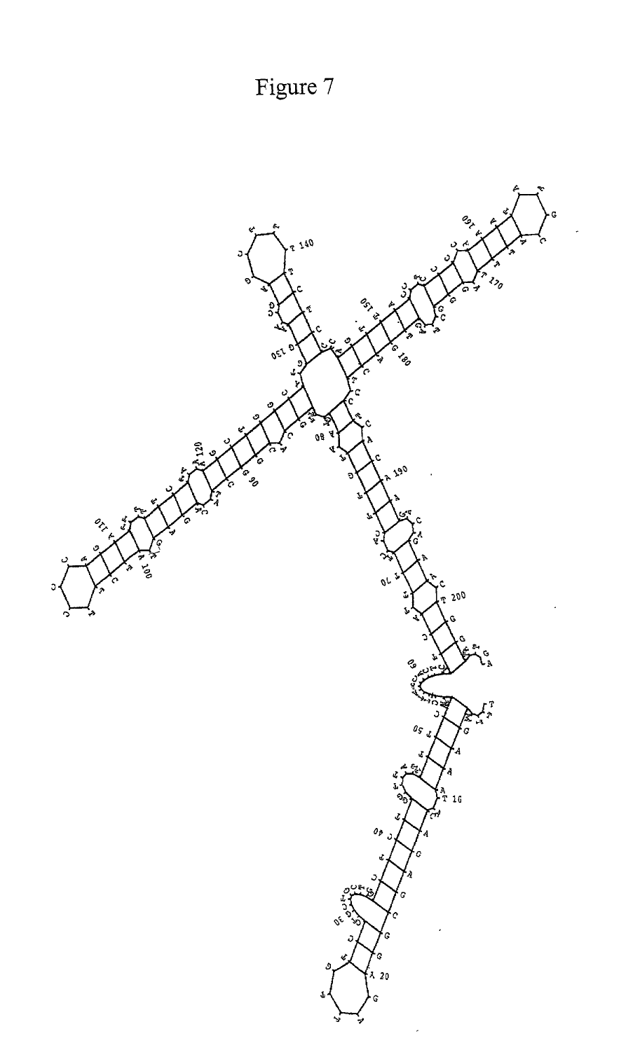

The nucleic acids of the invention can also have a secondary structure corresponding to that shown in FIGS. 7 to 9 comprising a 3-pronged stem-loop structure or a similar secondary structure having an energy of −40.8 kcal/mol or less, preferably −41.7 kcal/mol or less and more preferably −41.8 kcal/mol when analyzed according to the MFOLD program available from the University of Wisconsin, USA at (http://www.qcq.com) using a folding temperature of 37° C., a maximum size of interior loop of 30 and maximum loopsideness of an interior loop of 30 and nucleotide sequence in the form of a DNA molecule having nucleotides that correspond to the nucleotide sequence from 412 to 618 of SEQ ID NO:1.

Other nucleic acid molecules with a secondary structure according to the invention are obtainable by selecting two nucleotides in a naturally occurring RNA or DNA molecule encoding a member of the CD83 family of proteins, for example the CD83 nucleic acid sequence, that are base paired in a manner shown in FIGS. 7 to 9, 16 or 17 and then substituting these nucleotides in a pair-wise fashion such that the substitute nucleotides are based paired. Thus, in RNA molecules of the invention, a paired C-G nucleotide can be substituted by a G-C pair, an A-U pair or a U-A pair, a G-C base pair can be substituted by a C-G pair, an A-U pair or a U-A pair, an A-U base pair can be substituted by a U-A pair, a C-G pair or a G-C pair, and a U-A base pair can be substituted by a A-U pair, a C-G pair or a G-C pair. Likewise, in DNA molecules of the invention, a paired C-G nucleotide can be substituted by a G-C pair, an A-T pair or a T-A pair, a G-C base pair can be substituted by a C-G pair, an A-T pair or a T-A pair, an A-T base pair can be substituted by a T-A pair, a C-G pair or a G-C pair, and a T-A base pair can be substituted by a A-T pair, a C-G pair or a G-C pair. Preferably, C-G base pairs are substituted with G-C base pairs, A-T or A-U base pairs with T-A or U-A base pairs, respectively.

The nucleic acid molecules of the present invention can also be modified throughout their length and/or at the 5′ and/or 3′ end(s) to increase their stability.

Such modifications can be obtained by using α-anomers or 2′-O-alkyl ribosides (G. Zon, (1988) Pharmaceutical Research 5(9): 539-549; Helene, C. and Toulme, J.-J. (1990) Biochem. Biophys. Acta 1049: 99-125; Stein, C. A. and Cohen, J. S. (1988) Cancer Research 48:2659-2668). In addition, the phosphate backbone of the nucleic acid can be modified according to procedures known in the art to create nucleic acids of the invention comprising phosphothioate analogues, in which a non-bridging atom of the phosphate group is replaced by a sulfur atom, analogues in which a non-bridging atom of the phosphate group is replaced by a selenium atom, phosphorodithioate analogues, in which both non-bridging groups of the phosphate atom are replaced by sulfur, methylphosphonates, in which a non-bridging atom of the phosphate group is replaced by a methyl group, phosphoramidates, in which a non-bridging atom of the phosphate group is replaced by amide group substituted with one or two alkyl methyl or ethyl groups, and phosphotriesters, in which a non-bridging atom of the phosphate group is replaced by a methyl or ethyl ester. Further stabilizing modifications are described in Freier, S. M. and Altmann, K.-H., (1997) NAR 25(22): 4429-4443.

Furthermore, modifications to the nucleic acids of the inveniton include the addition of heterologous terminal sequences that form a terminal step-loop structure and therefore increase the stability of the cis-active sequence of the invention.

Furthermore, one or more of the bases of the nucleic acids can be replaced by modified bases. These modified bases include 2-methyladenine, 1-methylguanine, 5-methylcytosine, and 5-hydroxymethylcytosine.

The nucleic acids of the invention may also comprise non-nucleic acid components such as proteins that can be attached to the nucleic acids via a large number of known bifunctional linkers known in the art (for example, maleimidohexanoyl-N-hydroxysuccinimide ester and other linkers as described on pages 173 to 214 of the Pierce Products catalogue 1999/2000) and can aid in the cellular uptake of the nucleic acids. Examples for this are the tat-derived polypeptides as described by Vives, E. et al. (1997) J. Biol. Chem. 272(25): 16010-16017 and Bhorade, R. et al. (2000) Bioconjugate Chemistry 11(3): 301-305 and penetratin as described by Fischer, P. M. et al (2000) J. Pept. Res. 55(2): 163-172 and Bolton, S. J. et al. (2000) Eur. J. Neurosci. 12(8): 2847-2855.

Preferably, the above mentioned nucleic acids of the present invention are such that they do not contain regulatory sequences that lead to the expression of a polypeptide or protein from said nucleic acid in a cell.

In a further embodiment, compounds of the invention include protein molecules that specifically block the binding between a member of the HuR family of proteins and a mRNA encoding a member of the CD83 family of proteins and that reduce expression of a member of the CD83 family of proteins in a cell.

Thus, compounds of the invention comprise derivatives of members of the ELAV superfamily of proteins that specifically block the binding between a member of the HuR family of proteins and a mRNA encoding a member of the CD83 family of proteins and that reduce expression of a member of the CD83 family of proteins in a cell.

As defined herein, “members of the ELAV superfamily of proteins” include proteins comprising a partial amino acid sequence of ELAV (Robinow, S. et al. (1988) Science 242: 1570-1572, Genbank accession number M21152), (HuA=HuR (Ma, W.-J. et al. (1996) J. Biol. Chem. 271(14): 8144-8151), Genbank accession number U38175; HuB=Hel-N1 (King, P. H. et al. (1994) J. Neurosci. 14(4): 1943-1952, Genbank accession number U12431, SEQ ID NO:22 and 23); HuC (as deposited by Manley, T. and Furneaux, H. M. under Genbank accession number L26405, SEQ ID NO:24 and 25) and HuD (Szabo, A. et al. (1991) Cell 67(2): 325-333, Genbank accession number M62843, SEQ ID NO:26 and 27) as well as naturally occurring proteins encoded by the ELAV, HuA (=HuR), HuB (=Hel-N1), HuC and HuD genes that result from alternative gene expression, for example from alternate RNA splicing, such as HuDpro, HuDmex, Hel-N2 and HuC isoforms (See Liu, J. et al. (1995) Neurology 45:544-550 and Goa, F. C. (1994) PNAS (USA) 91:11207-11211). Furthermore, “members of the ELAV superfamily of proteins” include naturally occurring animal, for example zebrafish (Genbank accession numbers AF184245, AF184244, U62018, U17602, U17601 and U17600) preferably, mammal, protein homologues of the above proteins. For example, the Rel-N1 and Rel-N2 protein of rat (King, P. H. (1994) Gene 151(1-2): 261-265), rat “HuD” (protein sequence accession number 009032), rat ELAV-type RNA binding protein 3 (protein sequence accession number NP_{—}058893), mouse Mel-N1 (Genbank accession number NM_{—}010486), mouse “HuR” (Genbank accession number NM_{—}010485), mouse “HuD” (Genbank accession number NM_{—}010488) are encompassed within this definition.

Derivatives of a member of the ELAV superfamily of proteins according to the invention include members of the ELAV superfamily of proteins as defined above in which one or more amino acids has been added, deleted, substituted, inserted or inverted but that are still capable of specifically blocking the binding between a member of the HuR family of proteins and a mRNA encoding a member of the CD83 family of proteins and that reduce expression of a member of the CD83 family of proteins in a cell.

When one or more amino acids of a member of the ELAV superfamily of proteins is substituted to arrive at a derivative of a member of the ELAV superfamily of proteins, it is preferred that the one or more amino acids are conservatively substituted. For example, conservative substitutions include substitutions in which aliphatic amino acid residues such as Met, Ile, Val, Leu or Ala are substituted for one other. Likewise, polar amino acid residues can be substituted for each other such as Lys and Arg, Glu and Asp or Gln and Asn.

According to the invention, derivatives of a member of the ELAV superfamily of proteins also include derivatives in which one or more of the amino acids therein has an altered side chain. Such derivatized polypeptides include, for example, those comprising amino acids in which free amino groups form amine hydrochlorides, p-toluene sulfonyl groups, carobenzoxy groups; the free carboxy groups form salts, methyl and ethyl esters; free hydroxl groups that form O-acyl or O-alkyl derivatives as well as naturally occurring amino acid derivatives, for example, 4-hydroxyproline, for proline, 5-hydroxylysine for lysine, homoserine for serine, omithine for lysine etc. Also included are amino acid derivatives that can alter covalent bonding, for example, the disulfide linkage that forms between two cysteine residues that produces a cyclized polypeptide.

The length of the derivatives of a member of the ELAV superfamily of proteins according to the invention can vary as long as said derivative specifically blocks the binding between a member of the HuR family of proteins and a mRNA encoding a member of the CD83 family of proteins and reduces expression of a member of the CD83 family of proteins in a cell. Preferably, the derivatives of a member of the ELAV superfamily of proteins according to the invention are less than 250, more preferably 200 and most preferably 150 or less amino acids in length.

Preferably, a derivative of a member of the ELAV superfamily of proteins according to the invention does not contain a complete set of RRM1, RRM2 and RRM3 or the hinge region, including HNS.

For HuR, a complete RRM1 region is defined as the RNA Recognition Region from amino acid 19 to 100 of SEQ ID NO:6. For HuR, a complete RRM2 region is defined as the RNA Recognition Region from amino acid 103 to 189 of SEQ ID NO:6. For HuR, a complete RRM3 region is defined as the RNA Recognition Region from amino acid 245 to 326 of SEQ ID NO:6. Corresponding RRM1, RRM2 and RRM3 and hinge or HNS regions for other ELAV-like proteins can be deduced based on their homologies to the RRM1, RRM2 and RRM3 regions of HuR as defined above and preferably have 70%, more preferably, 80, and most preferably at least 90% homology to amino acids 19 to 100 of SEQ ID NO:6 for RRM1, amino acids 103 to 189 of SEQ ID NO:6 for RRM2, amino acids 245 to 326 of SEQ ID NO:6 for RRM3 and amino acids 190 to 244 of SEQ ID NO:6 for the hinge region, including amino acids 205 to 237 of SEQ ID NO:6 for HNS.

In a further embodiment, compounds of the invention comprise derivatives of protein ligands to HuR that specifically block the binding between a member of the HuR family of proteins and a mRNA encoding a member of the CD83 family of proteins and that reduce expression of a member of the CD83 family of proteins in a cell.

As defined herein, “protein ligands to HuR” include proteins comprising a partial amino acid sequence of SETS (Protein sequence accession number I59377), SETO (Protein sequence accession number A45018), pp32 (Genbank accession number U73477) and APRIL (Genbank accession number Y07969)as well as naturally occurring animal, preferably mammal, homologues of these proteins.

Derivatives of protein ligands to HuR according to the invention include SETU, SETH, pp32 and APRIL proteins as defined above in which one or more amino acids has been added, deleted, substituted, inserted or inverted but that are still capable of specifically blocking the binding between a member of the HuR family of proteins and a mRNA encoding a member of the CD83 family of proteins and that reduce expression of a member of the CD83 family of proteins in a cell.

When one or more amino acids of a protein ligand to HuR is substituted to arrive at a derivative of a member of the ELAV superfamily of proteins, it is preferred that the one or more amino acids are conservatively substituted. For example, conservative substitutions include substitutions in which aliphatic amino acid residues such as Met, Ile, Val, Leu or Ala are substituted for one other. Likewise, polar amino acid residues can be substituted for each other such as Lys and Arg, Glu and Asp or Gln and Asn.

According to the invention, protein ligands to HuR also include such proteins in which one or more of the amino acids therein has an altered side chain. Such derivatized proteins include, for example, those comprising amino acids in which free amino groups form amine hydrochlorides, p-toluene sulfonyl groups, carobenzoxy groups; the free carboxy groups form salts, methyl and ethyl esters; free hydroxl groups that form O-acyl or O-alkyl derivatives as well as naturally occurring amino acid derivatives, for example, 4-hydroxyproline, for proline, 5-hydroxylysine for lysine, homoserine for serine, omithine for lysine etc. Also included are amino acid derivatives that can alter covalent bonding, for example, the disulfide linkage that forms between two cysteine residues that produces a cyclized polypeptide.

The proteins according to the invention can be obtained using standard techniques for protein purification, for example, by chromatography (e.g., ion-exchange, size-exclusion, reverse-phase, immunoaffinity etc.). Other protein purification methods known in the art additionally can be used (see e.g., Deutscher et al., Guide to Protein Purification: Methods in Enzymology, Vol. 182, Academic Press, 1990). Alternatively, the proteins according to the inveniton can be obtained using recombinant expression methods as disclosed herein. For example, polynucleotide encoding the protein can be produced, inserted into a vector and transformed into host cells using well known techniques described herein and further known in the art (Sambrook et al., Molecular Cloning: A Laboratory Manual, Cold Spring Harbor Laboratory, New York,1989). Following transformation, protein may be isolated and purified in accordance with conventional methods. For example, lysate prepared from an expression host (e.g., bacteria) can be purified using HPLC, size-exclusion chromatography, gel electrophoresis, affinity chromatography, or other purification technique. Substantially pure proteins can also be obtained by chemical synthesis using a peptide synthesizer (e.g., Applied Biosystems, Inc., Foster City, Calif.; Model 430A or the like).

The present invention also relates to pharmaceutical compositions comprising the compounds of the present invention.

In addition, the present invention relates to the use of these pharmaceutical compositions for regulating an immune response involving the direct or indirect participation of DC. Preferably, the pharmaceutical compositions of the present invention are capable of reducing the expression of CD83 or a member of the CD83 family of proteins.

Moreover, the present invention relates to the use of the compounds of the present invention for the production of a pharmaceutical composition or medicament for regulating an immune response involving the direct or indirect participation of DC.

Furthermore, the invention relates to a method of treatment or prevention of disorders, diseases and syndromes involving the direct or indirect participation of DC by regulating an immune response, wherein an effective amount of a compound according to the invention or a pharmaceutical composition according to the invention comprising said compound is administered to a subject.

Thus, the compounds of the present invention can be used to inhibit CD83 protein expression and/or induction of the T cell stimulating mode of DC or induction of so-called “regulatory” T cells and thereby treat or prevent a variety of disorders, diseases and syndromes. “Regulatory” T cells are defined as IL-10-producing non-proliferating CD25⁺T cells.

For example, the compounds of the present invention can be used to modulate the growth, differentiation and/or activation of a variety of T cells such as cytotoxic T cells and helper T cells, the differentiation of helper T cells into Th1 cells or Th2 cells, the growth, stimulation and/or differentiation of B cells and treat or prevent disorders, diseases and syndromes caused by the failure of the body to regulate these processes in a healthy manner.

In addition, the compounds of the present invention can be used to treat or prevent rejection of tissue and/or organ transplants, particularly xenogenic tissue and/or organ transplants, that occurs as a result of for example graft-vs.-host disease or host-vs.-graft disease.

In a further embodiment of the present invention, the compounds of the present invention can be used to treat or prevent undesirable response to foreign antigens and therewith allergies and asthma or similar conditions.

Other disorders, diseases and syndromes that can be treated or prevented by the compounds of the present invention include autoimmune syndromes such as myasthemia gravis, multiple sclerosis and systemic lupus erythematosis, skin diseases such as psoriasis, rheumatoid arthritis and AIDS.

For therapeutic or prophylactic use, the compounds of the present invention are administered to a subject, preferably a mammal, more preferably a human patient, for treatment or prevention in a manner appropriate for the medical indication.

The production of pharmaceutical compositions with an amount of one or more compounds according to the invention and/or their use in the application according to the invention occurs in the customary manner by means of common pharmaceutical technology methods. For this, the compounds according to the invention are processed together with suitable, pharmaceutically acceptable adjuvents and/or carriers to medicinal forms suitable for the various indications and types of application. Thereby, the medicaments can be produced in such a manner that the respective desired release rate is obtained, for example a quick flooding and/or a sustained or depot effect.

Preparations for parenteral use, to which injections and infusions belong, are among the most important systemically employed medicaments for the above mentioned indications.

Preferably, injections are prepared either in the form of vials or also as so-called ready-to-use injection preparations, for example as ready-to-use syringes or single use syringes in addition to perforation bottles for multiple withdrawals. Administration of the injection preparations can occur in the form of subcutaneous (s.c.), intramuscular (i.m.), intravenous (i.v.), internodal (i.n.) or intracutaneous (i.c.) application. The respective suitable injection forms can especially be produced as solutions, crystal suspensions, nanoparticular or colloid-disperse systems, such as for example, hydrosols.

The injectable formulations can also be produced as concentrates which can be adjusted with aqueous isotonic dilution agents to the desired dosage of the compounds of the invention. Furthermore, they can also be produced as powders, such as for example lyophilisates, which are then preferably dissolved or dispersed immediately before application with suitable diluents. The infusions can also be formulated in the form of isotonic solutions, fat emulsions, liposome formulations, microemulsions and liquids based on mixed micells, for example, based on phospholipids. As with injection preparations, infusion formulations can also be prepared in the form of concentrates to dilute. The injectable formulations can also be applied in the form of continuous infusions as in stationary as well as in out-patient therapy, for example in the form of mini-pumps.

Albumin, plasma expanders, surface active compounds, organic solvents, pH influencing compounds, complex forming compounds or polymeric compounds can be added to the parenteral medicinal forms with the aim of decreasing the adsorption of the compounds of the present invention to materials such as injection instruments or packaging materials, for example plastic or glass.

The compounds according to the invention can be bound to nanoparticles in the preparations for parenteral use, for example on finely dispersed particles based on poly(meth)acrylates, polyacetates, polyglycolates, polyamino acids or polyether urethanes. The parenteral formulations can also be constructively modified as depot preparations, for example on the multiple unit principle, where the compounds of the present invention are incorporated in a most finely distributed and/or dispersed, suspended form or as crystal suspensions, or on the single unit principle, where the compounds according to the invention are enclosed in a medicinal form, for example, a tablet or a seed which is subsequently implanted. Often, these implantation or depot medicaments in single unit and multiple unit medicinal forms consist of so-called biodegradable polymers, such as for example, polyether urethanes of lactic and glycolic acid, polyether urethanes, polyamino acids, poly(meth)acrylates or polysaccharides.

Sterilized water, pH value influencing substances, such as for example organic and inorganic acids or bases as well as their salts, buffer substances for setting the pH value, agents for isotonicity, such as for example sodium chloride, monosodium carbonate, glucose and fructose, tensides and/or surface active substances and emulsifiers, such as for example, partial fatty acid esters of polyoxyethylene sorbitan (Tween®) or for example fatty acid esters of polyoxethylene (Cremophor®), fatty oils such as for example peanut oil, soybean oil and castor oil, synthetic fatty acid esters, such as for example ethyl oleate, isopropyl myristate and neutral oil (Miglyol®) as well as polymer adjuvents such as for example gelatin, dextran, polyvinylpyrrolidone, organic solvent additives which increase solubility, such as for example propylene glycol, ethanol, N,N-dimethylacetamide, propylene glycol or complex forming compounds such as for example citrates and urea, preservatives, such as for example hydroxypropyl benzoate and hydroxymethyl benzoate, benzyl alcohol, anti-oxidants, such as for example sodium sulfite and stabilizers, such as for example EDTA, are suitable as adjuvents and carriers in the production of preparations for parenteral use.

In suspensions, addition of thickening agents to prevent the settling of the compounds of the present invention from tensides and peptizers, to secure the ability of the sediment to be shaken, or complex formers, such as EDTA, ensues. This can also be achieved with the various polymeric agent complexes, for example with polyethylene glycols, polystyrol, carboxymethylcellulose, Pluronics® or polyethylene glycol sorbitan fatty acid esters. The compounds according to the invention can also be incorporated in liquid formulations in the form of inclusion compounds, for example with cyclodextrins. As further adjuvents, dispersion agents are also suitable. For production of lyophilisates, builders are also used, such as for example mannite, dextran, saccharose, human albumin, lactose, PVP or gelatin varieties.

A further systemic application form of importance is peroral administration as tablets, hard or soft gelatin capsules, coated tablets, powders, pellets, microcapsules, oblong compressives, granules, chewable tablets, lozenges, gums or sachets. These solid peroral administration forms can also be prepared as sustained action and/or depot systems. Among these are medicaments with an amount of one or more micronized compounds of the present invention, diffusions and erosion forms based on matrices, for example by using fats, wax-like and/or polymeric compounds, or so-called reservoir systems. As a retarding agent and/or agent for controlled release, film or matrix forming substances, such as for example ethylcellulose, hydroxypropylmethylcellulose, poly(meth)acrylate derivatives (for example Eudragit®), hydroxypropylmethylcellulose phthalate are suitable in organic solutions as well as in the form of aqueous dispersions. In this connection, so-called bio-adhesive preparations are also to be named in which the increased retention time in the body is achieved by intensive contact with the mucus membranes of the body. An example of a bio-adhesive polymer is the group of Carbomers®.

For sublingual application, compressives, such as for example non-disintegrating tablets in oblong form of a suitable size with a slow release of the compounds of the present invention, are especially suitable. For purposes of a targeted release of compounds of the present invention in the various sections of the gastrointestinal tract, mixtures of pellets which release at the various places are employable, for example mixtures of gastric fluid soluble and small intestine soluble and/or gastric fluid resistant and large intestine soluble pellets. The same goal of releasing at various sections of the gastrointestinal tract can also be conceived by suitably produced laminated tablets with a core, whereby the coating of the agent is quickly released in gastric fluid and the core of the agent is slowly released in the small intestine milieu. The goal of controlled release at various sections of the gastrointestinal tract can also be attained by multilayer tablets. The pellet mixtures with differentially released agent can be filled into hard gelatin capsules.

Anti-stick and lubricant and separating agents, dispersion agents such as flame dispersed silicone dioxide, disintegrants, such as various starch types, PVC, cellulose esters as granulating or retarding agents, such as for example wax-like and/or polymeric compounds on the basis of Eudragit®, cellulose or Cremophor® are used as a further adjuvents for the production of compressives, such as for example tablets or hard and soft gelatin capsules as well as coated tablets and granulates.

Anti-oxidants, sweetening agents, such as for example saccharose, xylite or mannite, masking flavors, aromatics, preservatives, colorants, buffer substances, direct tableting agents, such as for example microcrystalline cellulose, starch and starch hydrolysates (for example Celutab®), lactose, polyethylene glycols, polyvinylpyrrolidone and dicalcium phosphate, lubricants, fillers, such as lactose or starch, binding agents in the form of lactose, starch varieties, such as for example wheat or corn and/or rice starch, cellulose derivatives, for example methylcellulose, hydroxypropylcellulose or silica, talcum powder, stearates, such as for example magnesium stearate, aluminum stearate, calcium stearate, talc, siliconized talc, stearic acid, acetyl alcohol and hydrated fats are used.

In this connection, oral therapeutic systems constructed especially on osmotic principles, such as for example GIT (gastrointestinal therapeutic system) or OROS (oral osmotic system), are also to be mentioned.

Effervescent tablets or tabs, both of which represent immediately drinkable instant medicinal forms which are quickly dissolved or suspended in water are among the perorally administratable compressives. Among the perorally administratable forms are also solutions, for example drops, juices and suspensions, which can be produced according to the above given method, and can still contain preservatives for increasing stability and optionally aromatics for reasons of easier intake, and colorants for better differentiation as well as antioxidants and/or vitamins and sweeteners such as sugar or artificial sweetening agents. This is also true for inspisated juices which are formulated with water before ingestion. Ion exchange resins in combination with one or more compounds of the present invention are also to be mentioned for the production of liquid ingestable forms.

A special release form consists in the preparation of so-called floating medicinal forms, for example based on tablets or pellets which develop gas after contact with body fluids and therefore float on the surface of the gastric fluid. Furthermore, so-called electronically controlled release systems can also be formulated by which release of the compounds of the present invention can be selectively adjusted to individual needs.

A further group of systemic administration and also optionally topically effective medicinal forms are represented by rectally applicable medicaments. Among these are suppositories and enema formulations. The enema formulations can be prepared based on tablets with aqueous solvents for producing this administration form. Rectal capsules can also be made available based on gelatin or other carriers.

Hardened fat, such as for example Witepsol®, Massa Estarinum®, Novata®, coconut fat, glycerol-gelatin masses, glycerol-soap-gels and polyethylene glycols are suitable as suppository bases.

For long-term application with a systematic release of the compounds of the present invention up to several weeks, pressed implants are suitable which are preferably formulated on the basis of so-called biodegradable polymers.

As a further important group of systemically active medicaments, transdermal systems are also to be emphasized which distinguish themselves, as with the above-mentioned rectal forms, by circumventing the liver circulation system and/or liver metabolism. These plasters can be especially prepared as transdermal systems which are capable of releasing the compounds of the present invention in a controlled manner over longer or shorter time periods based on different layers and/or mixtures of suitable adjuvents and carriers. Aside from suitable adjuvents and carriers such as solvents and polymeric components, for example based on Eudragit®, membrane infiltration increasing substances and/or permeation promoters, such as for example oleic acid, Azone®, adipinic acid derivatives, ethanol, urea, propylglycol are suitable in the production of transdermal systems of this type for the purpose of improved and/or accelerated penetration.

As topically, locally or regionally administration medicaments, the following are suitable as special formulations: vaginally or genitally applicable emulsions, creams, foam tablets, depot implants, ovular or transurethral administration installation solutions. For opthalmological application, highly sterile eye ointments, solutions and/or drops or creams and emulsions are suitable.

In the same manner, corresponding otological drops, ointments or creams can be designated for application to the ear. For both of the above-mentioned applications, the administration of semi-solid formulations, such as for example gels based on Carbopols® or other polymer compounds such as for example polyvinylpyrolidone and cellulose derivatives is also possible.

For customary application to the skin or also to the mucus membrane, normal emulsions, gels, ointments, creams or mixed phase and/or amphiphilic emulsion systems (oil/water-water/oil mixed phase) as well as liposomes and transfersomes can be named. Sodium algenate as a gel builder for production of a suitable foundation or celluolose derivatives, such as for example guar or xanthene gum, inorganic gel builders, such as for example aluminum hydroxides or bentonites (so-called thixotropic gel builder), polyacrylic acid derivatives, such as for example Carbopol®, polyvinylpyrolidone, microcrystalline cellulose or carboxymethylcellulose are suitable as adjuvents and/or carriers. Furthermore, amphiphilic low and high molecular weight compounds as well as phospholipids are suitable. The gels can be present either as hydrogels based on water or as hydrophobic organogels, for example based on mixtures of low and high molecular paraffin hydrocarbons and vaseline.

Anionic, cationic or neutral tensides can be employed as emulsifiers, for example alkalized soaps, methyl soaps, amine soaps, sulfanated compounds, cationic soaps, high fatty alcohols, partial fatty acid esters of sorbitan and polyoxyethylene sorbitan, for example lanette types, wool wax, lanolin, or other synthetic products for the production of oil/water and/or water/oil emulsions.

Hydrophilic organogels can be formulated, for example, on the basis of high molecular polyethylene glycols. These gel-like forms are washable. Vaseline, natural or synthetic waxes, fatty acids, fatty alcohols, fatty acid esters, for example as mono-, di-, or triglycerides, paraffin oil or vegetable oils, hardened castor oil or coconut oil, pig fat, synthetic fats, for example based on acrylic, caprinic, lauric and stearic acid, such as for example Softisan® or triglyceride mixtures such as Miglyol® are employed as lipids in the form of fat and/or oil and/or wax-like components for the production of ointments, creams or emulsions.

Osmotically effective acids and bases, such as for example hydrochloric acid, citric acid, sodium hydroxide solution, potassium hydroxide solution, monosodium carbonate, further buffer systems, such as for example citrate, phosphate, Tris-buffer or triethanolamine are used for adjusting the pH value.

Preservatives, for example such as methyl- or propyl benzoate (parabenes) or sorbic acid can be added for increasing stability.

Pastes, powders or solutions are to be mentioned as further topically applicable forms. Pastes often contain lipophilic and hydrophilic auxiliary agents with very high amounts of fatty matter as a consistency-giving base.

Powders or topically applicable powders can contain for example starch varieties such as wheat or rice starch, flame dispersed silicon dioxide or silica, which also serve as diluents, for increasing flowability as well as lubricity as well as for preventing agglomerates.

Nose drops or nose sprays serve as nasal application forms. In this connection, nebulizers or nose creams or ointments can come to use.

Furthermore, nose spray or dry powder formulations as well as controlled dosage aerosols are also suitable for systemic administeration of the compounds of the present invention.

These pressure and/or controlled dosage aerosols and dry powder formulations can be inhaled and/or insufflated. Administration forms of this type also certainly have importance for direct, regional application in the lung or bronchi and larynx. Thereby, the dry powder compositions can be formulated for example as invention compound-soft pellets, as an invention compound-pellet mixture with suitable carriers, such as for example lactose and/or glucose. For inhalation or insufflation, common applicators are suitable which are suitable for the treatment of the nose, mouth and/or pharynx. The compounds of the present invention can also be applied by means of an ultrasonic nebulizing device. As a propellant gas for aerosol spray formulations and/or controlled dosage aerosols, tetrafluoroethane or HFC 134a and/or heptafluoropropane or HFC 227 are suitable, wherein non-fluorinated hydrocarbons or other propellants which are gaseous at normal pressure and room temperature, such as for example propane, butane or dimethyl ether can be preferred. Instead of controlled dosage aerosols, propellant-free, manual pump systems can also be used.

The propellant gas aerosols can also suitably contain surface active adjuvents, such as for example isopropyl myristate, polyoxyethylene sorbitan fatty acid ester, sorbitan trioleate, lecithins or soya lecithin.

In addition, when the pharmaceutical composition of the present invention comprises a nucleic acid of the invention for administration to a certain species of animal, the nucleic acid of the invention is preferably derived from that species. For example, when the pharmaceutical composition of the present invention is to be administered to humans, the nucleic acid of the pharmaceutical preferably comprises the coding region of a member of the CD83 family of proteins or a derivative thereof.

The nucleic acids of the invention can be administered in conjunction with agents that increase cell membrane permability and/or cellular uptake of the nucleic acids. Examples of these agents are polyamines as described for example by Antony, T. et al. (1999) Biochemistry 38: 10775-10784; branched polyamines as described for example by Escriou, V. et al (1998) Biochem. Biophys. Acta 1368(2): 276-288; polyaminolipids as described for example by Guy-Caffey, J. K. et al. (1995) J. Biol. Chem. 270(52): 31391-31396; DOTMA as desribed by Felgner, P. L. et al. (1987) PNAS USA 84(21): 7413-7417 and cationic porphyrins as described for example by Benimetskaya, L. et al. (1998) NAR 26(23): 5310-5317.

A nucleic acid molecule according to the inveniton or a nucleic acid that encodes a protein according to the invention may be inserted into a vector. The term “vector” refers to a plasmid, virus or other vehicle known in the art that can be manipulated by insertion or incorporation of a polynucleotide. Such vectors can be used for genetic manipulation (i.e., “cloning vectors”) or can be used to transcribe or translate the inserted polynucleotide (“expression vectors”). A vector generally contains at least an origin of replication for propagation in a cell and a promoter. Control elements, including expression control elements as set forth herein, present within an expression vector are included to facilitate proper transcription and translation (e.g., splicing signal for introns, maintenance of the correct reading frame of the gene to permit in-frame translation of mRNA and, stop codons etc.). The term “control element” is intended to include, at a minimum, one or more components whose presence can influence expression, and can also include additional components, for example, leader sequences and fusion partner sequences.

As used herein, the term “expression control element” refers to one or more nucleic acid sequences that regulate the expression of a nucleic acid sequence to which it is operatively linked. An expression control element operatively linked to a nucleic acid sequence controls transcription and, as appropriate, translation of the nucleic acid sequence. Thus an expression control element can include, as appropriate, promoters, enhancers, transcription terminators, a start codon (e.g., ATG) in front of a protein-encoding gene. “Operatively linked” refers to a juxtaposition wherein the components so described are in a relationship permitting them to function in their intended manner.

By “promoter” is meant a minimal sequence sufficient to direct transcription. Both constitutive and inducible promoters are included in the invention (see e.g., Bitter et al., Methods in Enzymology 153:516-544, 1987). Inducible promoters are activated by external signals or agents. Also included in the invention are those promoter elements which are sufficient to render promoter-dependent gene expression controllable for specific cell-types, tissues or physiological conditions; such elements may be located in the 5′, 3′ or intronic regions of the gene. Promoters useful in the invention also include conditional promoters. A “conditional promoter” is a promoter which is active only under certain conditions. For example, the promoter may be inactive or repressed when a particular agent, such as a chemical compound, is present. When the agent is no longer present, transcription is activated or derepressed.

Thus, when cloning in bacterial systems, constitutive promoters such as T7 and the like, as well as inducible promoters such as p1, of bacteriophage X, plac, ptrp, ptac (ptrp-lac hybrid promoter) may be used. When cloning in mammalian cell systems, constitutive promoters such as SV40, RSV, CMV including CMV-IE, and the like or inducible promoters derived from the genome of mammalian cells (e.g., metallothionein promoter) or from mammalian viruses (e.g., the mouse mammary tumor virus long terminal repeat; the adenovirus late promoter) may be used. Promoters produced by recombinant DNA or synthetic techniques may also be used to provide for transcription of the nucleic acid sequences of the invention.

Mammalian expression systems which utilize recombinant viruses or viral elements to direct expression may be engineered. For example, when using adenovirus expression vectors, nucleic acid of interest may be ligated to an adenovirus transcription/translation control complex, e.g., the late promoter and tripartite leader sequence. Alternatively, the vaccinia virus 7.5K promoter may be used.

Of particular interest are vectors based on bovine papilloma virus (BPV) which have the ability to replicate, as extrachromosomal elements. Shortly after entry of an extrachromosomal vector into mouse cells, the vector replicates to about 100 to 200 copies per cell. Because transcription of the inserted cDNA does not require integration of the plasmid into the host's chromosome, a high level of expression occurs. These vectors can be used for stable expression by including a selectable marker in the plasmid, such as the neo gene, for example. Alternatively, the retroviral genome can be modified for use as a vector capable of introducing and directing the expression of the nucleic acid of interest in host cells. High level expression may also be achieved using inducible promoters, including, but not limited to, the metallothionein RA promoter and heat shock promoters.

In yeast, a number of vectors containing constitutive or inducible promoters may be used. A constitutive yeast promoter such as ADH or LEU2 or an inducible promoter such as GAL may be used. Alternatively, vectors that facilitate integration of foreign nucleic acid sequences into a yeast chromosome, via homologous recombination for example, are known in the art and can be used.

A nucleic acid of interest according to the present invention may be inserted into an expression vector for expression in vitro (e.g., using in vitro transcription/translation assays or commercially available kits), or may be inserted into an expression vector that contains a promoter sequence which facilitates transcription and/or translation in either prokaryotes or eukaryotes (e.g., an insect cell) by transfer of an appropriate nucleic acid into a suitable cell. A cell into which a vector can be propagated and its nucleic acid transcribed, or encoded polypeptide expressed, is referred to herein as a “host cell” The term also includes any progeny of the subject host cell. Moreover, a nucleic acid of interest according to the present invention may be inserted into an expression vector for expression in vivo for somatic gene therapy for example. With these vectors, for example, retroviral vectors, lentivirus vectors, Adenovirus vectors, Adeno-associated virus vectors, plasmid expression vectors, the nucleic acids of the invention are expressed upon infection/introduction of the vector into DC.

Host cells include but are not limited to microorganisms such as bacteria, yeast, insect and mammalian organisms. For example, bacteria transformed with recombinant bacteriophage nucleic acid, plasmid nucleic acid or cosmid nucleic acid expression vectors containing a nucleic acid of interest; yeast transformed with recombinant yeast expression vectors containing a nucleic acid of interest; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a nucleic acid of interest; insect cell systems infected with recombinant virus expression vectors (e.g., baculovirus) containing a nucleic acid of interest; or animal cell systems infected with recombinant virus expression vectors (e.g., retroviruses, lentivirus, adenovirus, vaccinia virus) containing a nucleic acid of interest, or transformed animal cell systems engineered for stable expression.

For long-term expression of invention polypeptides in host cells, stable expression is preferred. Thus, using expression vectors which contain viral origins of replication, for example, cells can be transformed with a nucleic acid of interest controlled by appropriate control elements (e.g., promoter/enhancer sequences, transcription terminators, polyadenylation sites, etc.). Optionally, the expression vector also can contain a nucleic acid encoding a selectable or identifiable marker conferring resistance to a selective pressure thereby allowing cells having the vector to be identified, grown and expanded. Alternatively, the selectable marker can be on a second vector that is cotransfected into a host cell with a first vector containing an invention polynucleotide.

A number of selection systems may be used, including, but not limited to the herpes simplex virus thymidine kinase gene, hypoxanthine-guanine phosphoribosyltransferase gene, and the adenine phosphoribosyltransferase genes can be employed in tk-, hgprt or aprt cells respectively. Additionally, antimetabolite resistance can be used as the basis of selection for dhfr, which confers resistance to methotrexate; the gpt gene, which confers resistance to mycophenolic acid; the neomycin gene, which confers resistance to the aminoglycoside G-418; and the hygromycin gene, which confers resistance to hygromycin. Additional selectable genes have been described, namely trpB, which allows cells to utilize indole in place of tryptophan; hisD, which allows cells to utilize histinol in place of histidine; and ODC (ornithine decarboxylase) which confers resistance to the omithine decarboxylase inhibitor, 2-(difluoromethyl)-DL-onithine, DFMO.

As used herein, the term “transformation” means a genetic change in a cell following incorporation of DNA exogenous to the cell. Thus, a “transformed cell” is a cell into which (or a progeny of which) a DNA molecule has been introduced by means of recombinant DNA techniques.

Transformation of a host cell with DNA may be carried out by conventional techniques known to those skilled in the art. For example, when the host cell is a eukaryote, methods of DNA transformation include, for example, calcium phosphate co-precipitates, conventional mechanical procedures such as microinjection, electroporation, insertion of a plasmid encased in liposomes, and viral vectors. Eukaryotic cells also can be cotransformed with DNA sequences encoding a nucleic acid of interest, and a second foreign DNA molecule encoding a selectable phenotype, such as the those described herein. Another method is to use a eukaryotic viral vector, such as simian virus 40 (SV40) or bovine papilloma virus, to transiently infect or transform eukaryotic cells and express the protein.

When the compound of the invention is an RNA molecule, said RNA molecule can be delivered to the cells of a subject by transforming said cells with an expression vector that comprises a nucleotide sequence encoding said RNA molecule under control of sequence that allows for the transcription of the nucleotide sequence.

Thus, the present invention relates to an expression vector comprising a nucleic acid sequence encoding an RNA according to the invention that specifically blocks the binding between a member of the HuR family of proteins and a mRNA encoding a member of the CD83 family of proteins and that reduces expression of a member of the CD83 family of proteins in a cell.

Preferably, the expression vector is constructed such that the RNA molecule is not translated into a polypeptide or protein in a cell.

Furthermore, the present invention relates to a host cell transformed with the above mentioned expression vector.

The present invention also relates to the use of an expression vector encoding an nucleic acid according to the invention that specifically blocks the binding between a member of the HuR family of proteins and a mRNA encoding a member of the CD83 family of proteins and that reduces expression of a member of the CD83 family of proteins in a cell for the production of a medicament for the treatment and prevention of disorders, diseases and syndromes involving the direct or indirect participation of DC by regulating an immune response.

Furthermore, the invention relates to a method of treatment or prevention of disorders, diseases and syndromes involving the direct or indirect participation of dendritic cells by regulating an immune response, wherein an effective amount of an expression vector encoding an nucleic acid according to the invention that specifically blocks the binding between a member of the HuR family of proteins and a mRNA encoding a member of the CD83 family of proteins and that reduces expression of a member of the CD83 family of proteins in a cell is administered to a subject.

When the compound of the invention is protein, said protein molecule can be delivered to the cells of a subject by transforming said cell with an expression vector that comprises a nucleotide sequence encoding said protein under control of sequence that allows for the transcription of the nucleotide sequence.

Thus, the present invention relates to an expression vector comprising a nucleic acid sequence encoding a derivative of a member of the ELAV superfamily of proteins or a derivative of a ligand to HuR that specifically blocks the binding between a member of the HuR family of proteins and a mRNA encoding a member of the CD83 family of proteins and that reduces expression of a member of the CD83 family of proteins in a cell.

Furthermore, the present invention relates to a host cell transformed with the above mentioned expression vector.

The present invention also relates to the use of an expression vector encoding a derivative of a member of the ELAV superfamily of proteins or a derivative of a ligand to HuR that specifically blocks the binding between a member of the HuR family of proteins and a mRNA encoding a member of the CD83 family of proteins and that reduces expression of a member of the CD83 family of proteins in a cell for the production of a medicament for treating any of the medical indications mentioned above.

Furthermore, the invention relates to a method of treatment or prevention of disorders, diseases and syndromes involving the direct or indirect participation of dendritic cells by regulating an immune response wherein an effective amount of an expression vector encoding a derivative of a member of the ELAV superfamily of proteins or a ligand to HuR that specifically blocks the binding between a member of the HuR family of proteins and a mRNA encoding a member of the CD83 family of proteins and that reduces expression of a member of the CD83 family of proteins in a cell is administered to a subject.

The present invention also relates to a method for screening and/or identifying compounds that block the binding between a member of the HuR family of proteins and a mRNA encoding a member of the CD83 family of proteins comprising the steps of incubating one or more compounds in a reaction comprising:

(a) a nucleic acid molecule that contains at least a portion of the coding region of a member of the CD83 family of proteins or a derivative thereof and

(b) a member of the ELAV superfamily of proteins or derivative thereof

under conditions sufficient to allow the components to interact and determining whether the compound blocks the binding between the nucleic acid molecule and the member of the ELAV superfamily of proteins.

As used herein, the term “incubating” refers to conditions that allow the contact, binding or interaction between (a) and (b) above and the test compound. The term “contacting” includes in solution, in solid phase and in cells.

Both the nucleic acid molecule that contains at least a portion of the coding region of a member of the CD83 family of proteins or a derivative thereof and the member of the ELAV superfamily of proteins or derivative thereof that are used in this method are defined as above.

A given compound can be considered to block the binding between the nucleic acid molecule and the member of the ELAV superfamily of proteins when said compound blocks the high-affinity and specific binding of HuR to the stem-loop structure of CD83 mRNA.

Compounds that block the binding between a member of the HuR family of proteins and a mRNA encoding a member of the CD83 family of proteins are identified by determining an activity of a protein from the CD83 family of proteins or the expression of a member of the CD83 family of proteins in the presence and in the absence of a test compound. An activity of a protein from the CD83 family of proteins or the expression of a member of the CD83 family of proteins, can be determined using cell free systems, in cells and in a whole organism. For example, electrophoretic mobility shift assays (EMSA) as described in the examples can be used to identify a compound that blocks the binding between a member of the HuR family of proteins and a mRNA encoding a member of the CD83 family of proteins. In cells, compounds that block the binding between a member of the HuR family of proteins and a mRNA encoding a member of the CD83 family of proteins can be identified by treating cells that express a mRNA encoding a member of the CD83 family of proteins with a test compound, and then examining the phenotype of said cells for functional expression of the member of the CD83 family of proteins.

Compounds that that block the binding between a member of the HuR family of proteins and a mRNA encoding a member of the CD83 family of proteins can be identified by detecting the expression of a mRNA comrising a reporter gene sequence linked to a portion of the coding region of a member of the CD83 family of proteins or a derivative thereof (i. e., a functional analysis as provided in the examples). The reporter provides a detection signal (e.g., the amount of transcript or protein product produced by the reporter gene) that corresponds to the degree of binding between a member of the HuR family of proteins and a mRNA encoding a member of the CD83 family of proteins. A compound “blocks” binding between a member of the HuR family of proteins and a mRNA encoding a member of the CD83 family of proteins if the detection signal provided by the reporter gene is decreased as compared with the signal in the absence of the test compound. A compound “inhibits” binding between a member of the HuR family of proteins and a mRNA encoding a member of the CD83 family of proteins if the signal is decreased as compared with the signal in the absence of the test compound.

The signal provided by the reporter gene can be, for example, RNA, protein, an enzymatic activity and the like. Thus, the signal can be detected by a variety of methods known in the art, including northern analysis, RNA dot blots, ELISA or RIA, Western blots, SDS-PAGE alone, or in combination with antibodies that immunoprecipitate the reporter gene product. Expressed products that provide an enzymatic activity or detection signal are preferred and include, for example, β-galactosidase, alkaline phosphatase, horseradish peroxidase, luciferase, green fluorescent protein and chloramphenicol acetyl transferase. Cells contemplated for use in these methods include the cells describe herein, for example, insect cells, mammalian cells (e.g., CV-1, COS, HeLa and L-cells) and yeast cells.

Test compounds that may effect binding between a member of the HuR family of proteins and a mRNA encoding a member of the CD83 family of proteins are found among biomolecules including, but not limited to: nucleic acids, proteins, peptides, polypeptides, peptidomimetics, saccharides, fatty acids, steroids, purines, pyrimidines, derivatives thereof, structural analogs thereof or combinations thereof. Test compounds further include chemical compounds (e.g., small organic molecules having a molecular weight of more than 50 and less than 5,000 Daltons, such as hormones). Candidate organic compounds comprise functional groups necessary for structural interaction with proteins or nucleic acids, particularly hydrogen bonding, and typically include at least an amine, carbonyl, hydroxyl or carboxyl group, preferably at least two of the functional chemical groups. The candidate organic compounds often comprise cyclical carbon or heterocyclic structures and/or aromatic or polyaromatic structures substituted with one or more of the above functional groups. Known pharmacological compounds are candidates that may further be subjected to directed or random chemical modifications, such as acylation, alkylation, esterification, amidification, etc., to produce structural analogs.

Test compounds can additionally be contained in libraries, for example, synthetic or natural compounds in a combinatorial library. Numerous libraries are commercially available or can be readily produced; means for random and directed synthesis of a wide variety of organic compounds and biomolecules, including expression of randomized oligonucleotides and oligopeptides, also are known. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Such libraries are useful for the screening of a large number of different compounds.

A variety of other compounds may be included in the screening method. These include agents like salts, neutral proteins, e.g., albumin, detergents, etc. that are used to facilitate optimal protein-protein, protein-nucleic acid or nucleic acid-nucleic acid binding or interactions and/or reduce nonspecific or background binding or interactions. For example, reagents that improve the efficiency of the assay, such as protease inhibitors, nuclease inhibitors, antimicrobial agents, etc., may be used. The mixture of components is added in any order that provides for the requisite modulation. Moreover, such test compounds additionally can be modified so as to facilitate their identification or purification. Such modifications are well known to the skilled artisan (e.g., biotin and streptavidin conjugated compounds).

Incubations are performed at any suitable temperature, typically between 4 and 40° C. Incubation periods are selected for optimum activity, but may also be chosen to facilitate rapid high-throughput screening. Typically, between 0.1 and 72 hours incubation time will be sufficient.

In preferred embodiments of the invention, the method for screening and/or identifying compounds that block the binding between a member of the HuR family of proteins and a mRNA encoding a member of the CD83 family of proteins can be carried out in the form of RNA gel shift assays, filter binding assays, Biacore interaction analysis (Biacore, Uppsala, Sweden), Scintillation Proximity Assay (Amersham Pharmacia Biotech, Freiburg, Germany), RNAse protection assays, cell-based RNA binding assays (see Blair et al. (1998) RNA 4: 215-225), yeast 3-hybrid assays (for example, RNA-Protein Hybrid Hunter® System (Invitrogen, Groningen, The Netherlands)), and reporter gene assays in eukaryotic cells as described above.

In the following, various aspects of the invention are more closely described via examples. However, the invention should not be considered as being to the examples.

### DETAILED DESCRIPTION OF THE INVENTION

### Example 1

RNA mobility shift experiments are a very sensitive method for characterizing RNA-protein interactions. For this, the RNA to be examined can be amplified and radioactively labeled with the aid of in vitro transcription from a linear DNA template. The labeling generally occurs with [α-⁼P]-UTP. In order to ensure the correct folding of the RNA, this is heated in a water bath to 80° C. and cooled to room temperature. RNA obtained in this manner is then incubated with the desired protein and subsequently analyzed on a native polyacrylamide gel, i.e. under non-denaturing conditions.

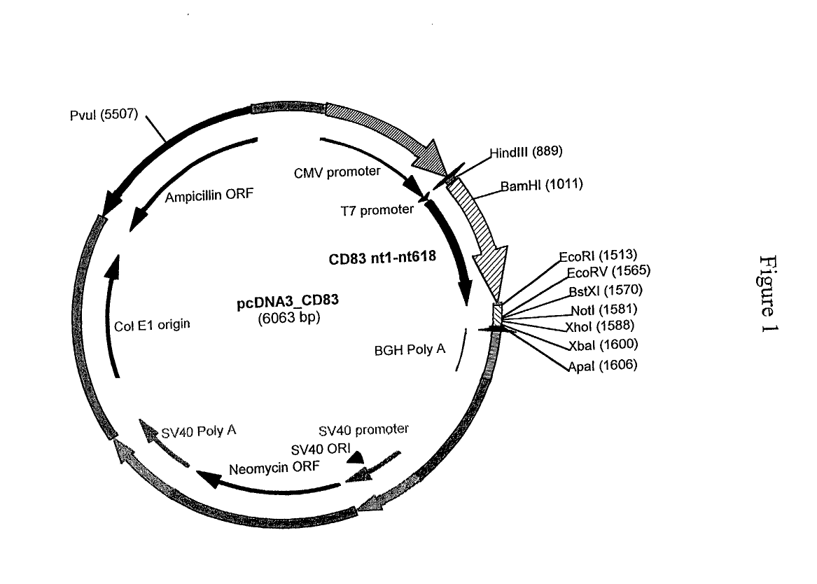

In order to examine whether a mRNA encoding a member of the CD83 family of proteins specifically binds to a member of the HuR family of proteins, a vector, pcDNA3-CD83, was constructed in which nucleotides 1 to 618 of SEQ ID NO:1 were inserted into the HindIII-EcoRI sites of the commercially available vector pcDNA3 (Invitrogen, Groningen, The Netherlands) under the control of a phage T7 promoter (FIG. 1).

In order to obtain radioactively labeled RNA for the RNA mobility shift experiments, approximately 20 μg of this vector were linearized with EcoRI and purified on a 0.8% agarose gel. After elution from the gel, the template was extracted with phenol and ethanol precipitated. The resulting DNA was resuspended at a concentration of 1.0 μg/ml in 20μl nuclease-free water. Radioactively labeled RNA was generated using a Riboprobe In Vitro Transcription System (Promega, Heidelberg, Germany) according to the instructions of the manufacturer using [α-³²P]-UTP. The transcription products were controlled for size and purity by separating 1 μl of the in vitro transcription reaction on a denaturing RNA agarose gel (1.2%), blotting the gel to a nitrocellulose membrane (Amersham-Pharmacia, Freiburg, Germany) for 8 hours and examining the nitrocellulose filter with the aid of photographic film using RNA size standards.

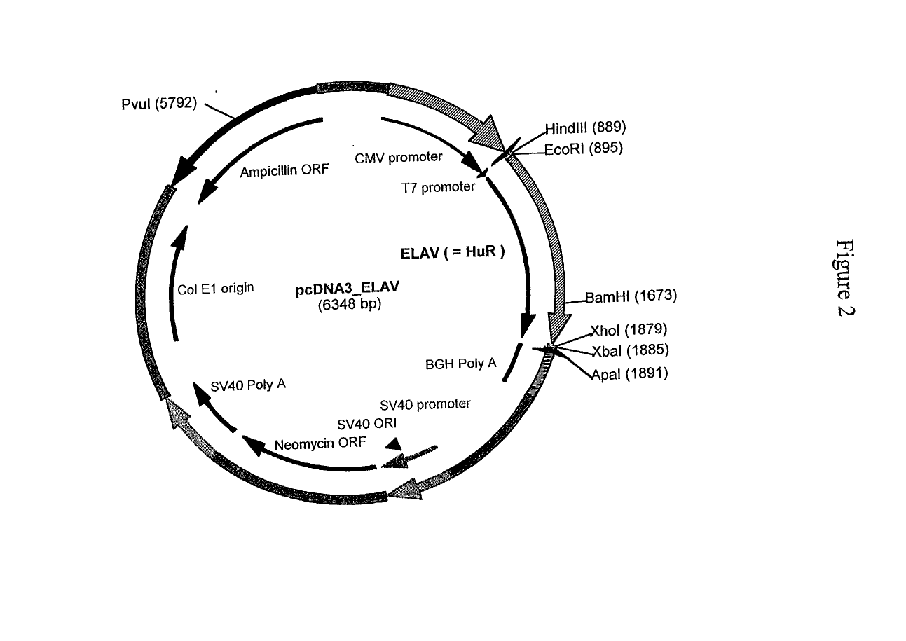

Plasmid pGEX5X-1-GST-ELAV encoding a recombinant GST-ELAV (HuR) fusion protein (GST: glutathione-S-transferase) was constructed by cloning nucleotides 1 to 981 of SEQ ID NO:5 into the EcoRI and XhoI sites of the commercially available plasmid pGEX-5X-1 (Amersham-Pharmacia, Freiburg, Germany) (FIG. 2).

A recombinant GST-ELAV (HuR) fusion protein was obtained by isolating the protein from E. coli BL21 (Stratagene GmbH, Heidelberg, Germany) transformed with plasmid pGEX5X-1-GST-ELAV. On the day before isolation, the bacterium was grown in a 50 ml pre-culture. On the following day, this pre-culture was diluted 1:10 in LB medium and incubated until an OD600 of approximately 0.5-0.6. The induction of fusion protein occurred by addition of IPTG (final concentration 1 mM). After 4 hours, the culture was cooled on ice and then centrifuged at 5000 rpm in a Sorvall GSA rotor for 10 min at 4° C. The pellet was then suspended in 10 ml of a buffer comprising 50 ml PBS, 100 [l PMSF, 50 Al leupeptin, 50μl pepstatin, 50 μl aprotein and 50 μl DNAse I. After addition of 26 μl of 1 M MnCl₂, 260 μl of 1 M MgCl₂ and a spatula tip of lysozyme, the suspension was briefly mixed and left on ice for 15 min. This was then sonicated 3 times for 10 sec. (2 impulses per second/max. output) and was adjusted to a final sodium chloride concentration of 0.5 M NaCl. To completely lyse the cells, Triton TX-100 was added to a concentration of 1% and the suspension was held on ice for 10 min. Finally the cell residue was centrifuged at 14,000 rpm in a Sorvall SS34 rotor for 30 min. at 4° C.

750 μl of glutathione-Sepharose (Amersham-Pharmacia, Freiburg, Germany) was washed 3 times with 10 ml of PBS. The supernatant of the bacterial cell extraction and the glutathione-Sepharose were mixed and incubated on a rotating shaker placed in a refrigerator for 1 hour. The glutathione-Sepharose, cell extract mixture was placed on a Bio-Prep column (BioRad, Munich, Germany) and washed 3 times each with 5 ml of a buffer consisting of 15 ml PBS 1 ml Triton TX-100 (20% stock solution) and 3 times each with a buffer consisting of 15 ml PBS and 50 μl PMSF. The elution of the GST-HuR fusion protein from the glutathione-Sepharose occurred in five elution steps each with 1 ml of a buffer comprising 50 mM Tris base, 150 mM NaCl and 15 mM glutathione. 1 ml of this elution buffer was added to the column, the column was closed and then incubated for 10 minutes on a rotating platform. The protein solutions were then combined and dialyzed for 12 hours against PBS.

RNA mobility shift experiments were then carried out using the radioactively labeled CD83 RNA and the GST-ELAV (HuR) fusion protein as described above. 1 μl of approximately 150-200 ng/ml radioactively labeled CD83 RNA and 1 μl of GST-ELAV (HuR) (protein concentration: 0.3 mg/ml) were used per reaction. Alternatively, GST protein without ELAV sequences was used as a control. The radioactively labeled CD83 RNA was heated to 80° C. in a water bath and cooled to room temperature directly before the reaction. The GST-ELAV (HuR) fusion protein (or GST control protein directly expressed from the vector pGEX-5X-1 was carefully mixed with a binding mixture consisting of 1 μl radioactively labeled CD83 RNA, 1 μl Rnasin® RNAse inhibitor (diluted 5:1 in PBS), 1 μl 10×PBS and 6 μl MS2 RNA or 5S RNA (Roche Diagnostics, Mannheim, Germany) (0.8 μg/μl) and incubated at room temperature for 30 min. 3 μl of 50% glycerin were added to each reaction to ease loading and analysis of the binding between the GST-ELAV (HuR) fusion protein and the CD83 RNA occurred on a 4% or 6% native polyacrylamide gel with a TBE running buffer of pH 8.3.

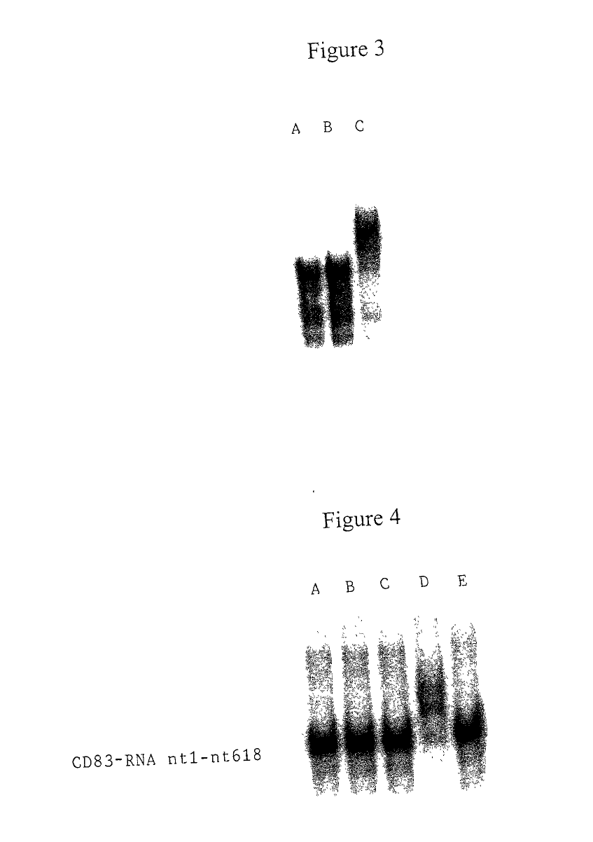

As can be seen from FIG. 3, the GST-ELAV (HuR) protein shifted the CD83 RNA to a higher position in the gel, whereas no shift occurred in the reaction comprising CD83 RNA and the GST protein control.

### Example 2

As a control for the specificity of the interaction between HuR and CD83 mRNA, RNA mobility shifts as described in Example 1 were performed using the GST protein as described above, a GST-L5 fusion protein (Schatz et al. (1998) PNAS USA 95: 1607-1612), and a GST- M9 fusion protein comprising the M9 domain of the heterogeneous nuclear ribonucleoprotein hnRNP A1 (Pollard, V. W. et al. (1996) Cell 86: 985-994).

As can be seen in FIG. 4, a shift in the mobility of the CD83 RNA is only observed with protein comprising the HuR sequence.

### Example 3

A standard method to test the specificity of a nucleic acid-protein interaction demonstrated in a RNA mobility shift experiment is to compete binding of the radioactively labeled RNA to the protein with unlabeled RNA. For this, the binding mixture as described above was produced with increasing amounts of non-radioactive CD83 RNA and the RNA mobility shift experiments were carried out as described above, except that the binding mixture was incubated with the respective protein for 15 min and then the same amount of radioactively labeled CD83 RNA (1 μl) was subsequently added to each reaction and incubation was continued for a further 15 min.

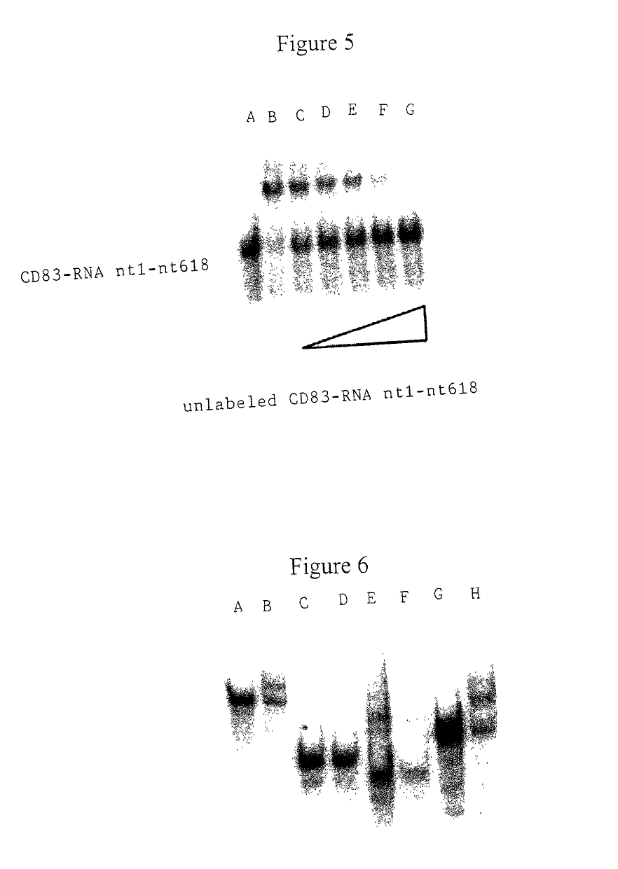

The results of this experiment are shown in FIG. 5. It was demonstrated that unlabeled CD83 RNA is capable of titrating free GST-ELAV (HuR) protein.

### Example 4

Having established that the binding of HuR to CD83 mRNA is specific, several experiments were conduced to determine and delineate the region of the CD83 mRNA to which HuR binds.

In order to delineate the region of CD83 mRNA to which HuR binds, the coding region of CD83 or sub-fragments of the CD83 coding sequence were cloned into the HindIII and/or EcoRI restriction sites in the multiple cloning site of the vector pcDNA3 under control of the phage T7 promoter. These sub-fragments were generated using a standard PCR reaction as described above and the following primers:

Sub-fragment CD83 nt1-nt294 (=aa1-aa98):

5′ primer; introduces HindIII site and start codon:

5′ -ATTTAAAAGCTTATGTCGCGCGGCCTCCalif.GCTTCTG-3′ (SEQ ID NO:7) and

3′ primer; introduces EcoRI site and stop codon:

5′ -ATTTAAGAATTCTCAGGTAGTGTTTCGGATCTTCAGGGAATA-3′ (SEQ ID NO:8);

Sub-fragment CD83 nt202-nt414 (aa68-aa138):

5′ primer; introduces HindIII site and start codon:

5′ -ATTTAAAAGCTTATGCTCAGGGGACAGCACTATCATCAGAAG-3′ (SEQ ID NO:9) and

3′ primer; introduces EcoRI site and stop codon:

5′ -ATTTAAGAATTCTCAAAAAGTCTCTTCTTTACGCTGTGCAGGGC-3′ (SEQ ID NO:10);

Sub-fragment CD83 nt295-nt618 (=aa99-aa205):

5′ primer; introduces HindIII site and start codon:

5′ -ATTTAAAAGCTTATGAGCTGCAACTCGGGGACATACAGG-3′ (SEQ ID NO:11) and

3′ primer; introduces EcoRI site:

5′ -ATTTAAGAATTCTCATACCAGTTCTGTCTTGTGAGGAGTCAC-3′ (SEQ ID NO:12).

The PCR reaction mix was composed of the following components:

10 μl template DNA (100 ng; 0.01 μg/μl)

10 μl 10×polymerase buffer (Roche Diagnostics, Mannheim, Germany)

10 μl dNTP mix (each NTP 2 mM)

2.5 μl primer 1 (0.1 μg/μl)

2.5 μl primer 2 (0.1 μg/μl)

64 μl water

1 μl PWO polymerase (Roche Diagnostics, Mannheim, Germany)

The PCR was carried out in a thermostable heating block with cover heating (Genius Thermocycler, Techne, N.J., USA). The amplification occurred in 45 cycles wherein the programmed cycles were as follows: the first cycle was performed with a denaturation step at 95° C. for 3 min., a step for hybridization of the primers at 52° C. for 2 min. and a synthesis reaction at 72° C. for 6 min.; the remaining 44 cycles were performed with a denaturation step at 95° C. for 1 min., a step for hybridization of the primers at 65° C. for 1 min. and a synthesis reaction at 72° C. for 4 min., whereby the time for each ensuing synthesis reaction was increased by 1 second; at the end of the program, a synthesis step was performed at 72° C. for 10 min.

The resulting vectors were linearized with EcoRI and radioactively labeled RNA was produced and employed in RNA mobility shift assays as described above.

The results are shown in FIG. 6 and demonstrate that a region of HuR binding to CD83 mRNA is found from nucleotide 415 to nucleotide 618 of (SEQ ID NO:1).

### Example 5

The secondary structure of a nucleic acid can be calculated at a certain temperature with the aid of the program MFOLD of the Genetics Computer Group (GCG) of the University of Wisconsin, USA (http://www.qcq.com).

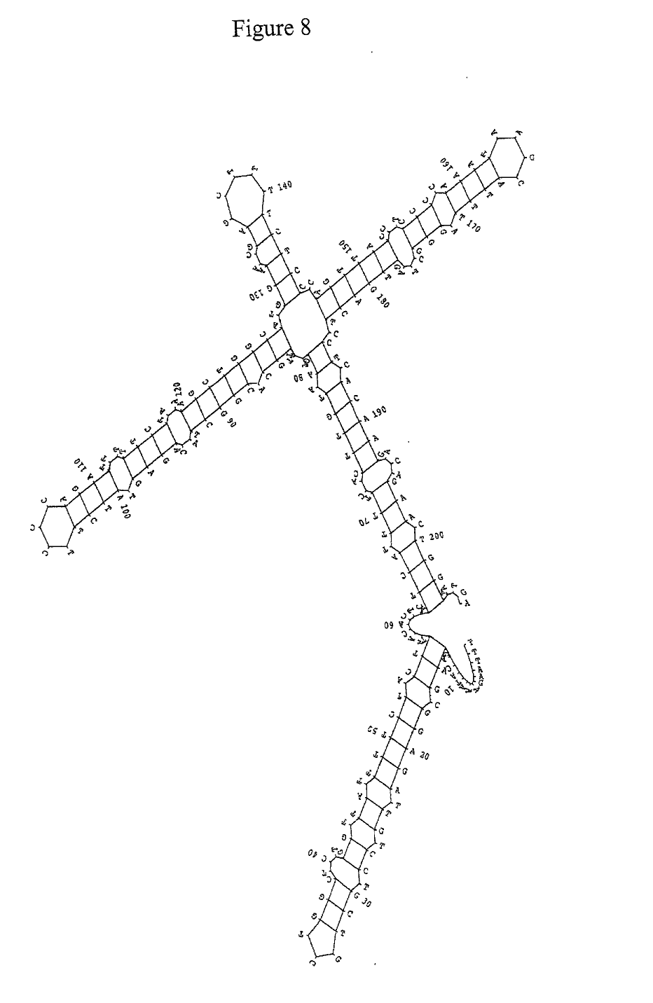

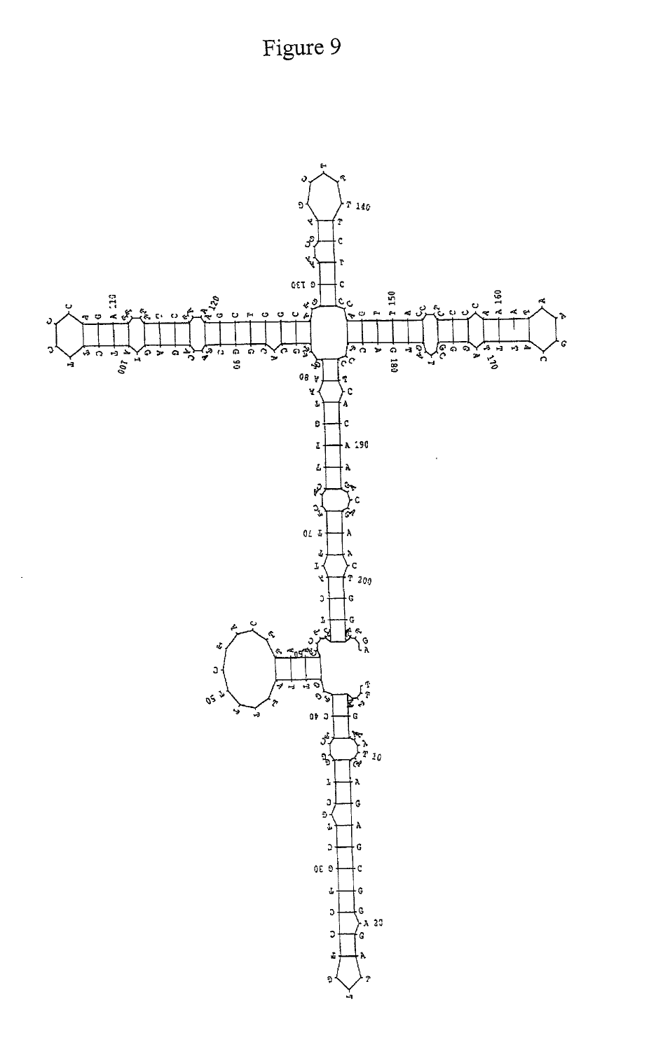

A DNA sequence containing nucleotides 412 to 618 of SEQ ID NO:1 encoding the amino acids 138-205 of CD83 was used as input data in the MFOLD program to calculate the secondary structure of the corresponding RNA molecule. A folding temperature of 37° C., a maximum size of interior loop of 30 and maximum loopsideness of an interior loop of 30 was used. The most probable secondary structure based on this calculation, i.e. the secondary structure having the most negative energy in kcal/mol is presented in FIG. 7 (−41.8 kcal/mol); the second most probable in FIG. 8(−41.7 kcal/mol) and the third most probable in FIG. 9(−40.8 kcal/mol).

Each of these secondary structures has two clearly recognizable domains: a first domain (stem-loop-1) comprising a single stem-loop structure and containing the CD83 RNA sub-fragment from nucleotide 412 to nucleotide 465 of SEQ ID NO:1 and a further, second domain (stem-loop-2) comprising a single 3-pronged stem-loop structure and containing the CD83 RNA sub-fragment from nucleotide 466 to nucleotide 618 of SEQ ID NO:1.

### Example 6

In order to further characterize the HuR binding domain on the CD83 mRNA, the first domain.

In order to further delineate the region of CD83 mRNA to which HuR binds, the domain stem-loop-1 comprising a single stem-loop structure and containing the CD83 RNA sub-fragment from nucleotide 295 to nucleotide 465 of SEQ ID NO:1 and the stem-loop-2 domain comprising a single 3-pronged stem-loop structure and containing the CD83 RNA sub-fragment from nucleotide 466 to nucleotide 618 of SEQ ID NO:1 were cloned into the HindIII and/or EcoRI restriction sites in the multiple cloning site of the vector pcDNA3 under control of the phage T7 promoter. These sub-fragments were generated using a standard PCR reaction as described above and the following primers:

Sub-fragment CD83 nt295-nt465 (= aa99-aa155):
5′ primer; introduces HindIII site and
start codon:
5′-ATTTAAAAGCTTATGAGCTGCAACTCGGGGACATACAGG-3′
(SEQ ID NO:11) and
3′ primer; introduces EcoRI site and stop codon:
5′-ATTTAAGAATTCTCAGTAGAAAATAACCAGAGCCAGCAGCAGG-3′
(SEQ ID NO:13);
Sub-fragment CD83 nt466-nt618 (aa156-aa205):
5′ primer; introduces HindIII site and
start codon:
5′-ATTTAAAAGCTTATGTTAACACTCATCATTTTCACTTGTAAGTTTGC
-3′ (SEQ ID NO:14) and
3′ primer; introduces EcoRI site:
5′-ATTTAAGAATTCTCATACCAGTTCTGTCTTGTGAGGAGTCAC-3′
(SEQ ID NO:12).

The resulting vectors were linearized with EcoRI and radioactively labeled RNA was produced and employed in RNA mobility shift assays as described above.

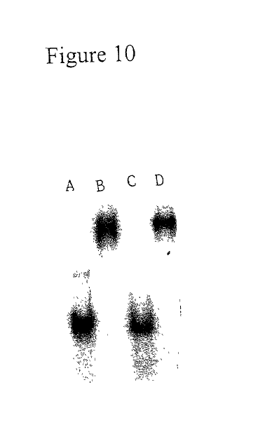

The results are shown in FIG. 10 and demonstrate that HuR can bind to both the stem-loop-1 and stem-loop-2 domain of the CD83 RNA.

### Example 7

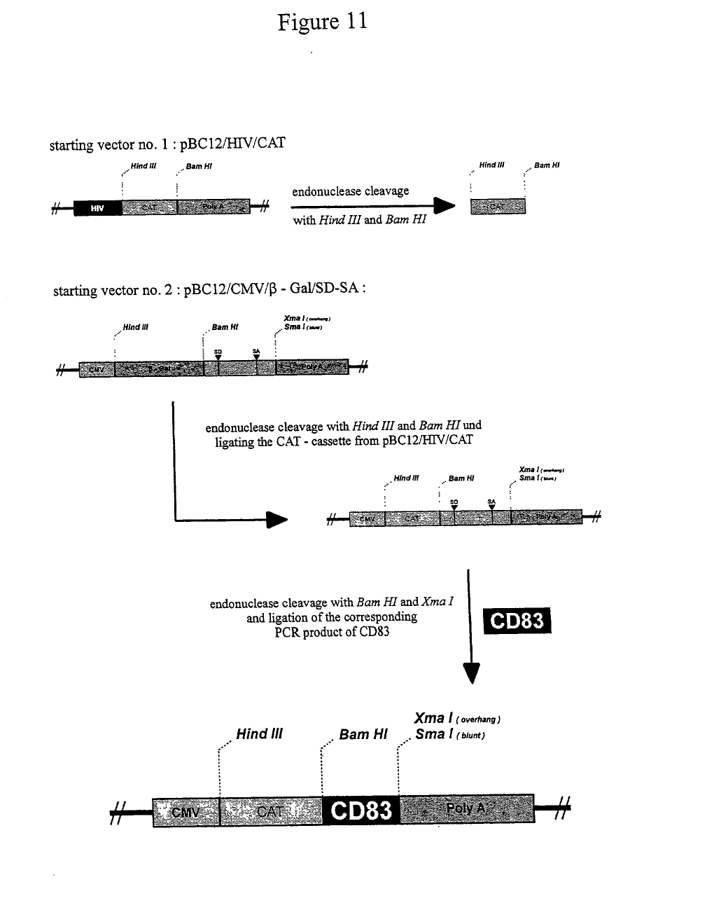

In order to determine whether the binding of HuR to CD83 mRNA leads to a biologically significant effect, a reporter plasmid, pB12/CMV/CAT, was constructed by cloning the chloramphenicol transferase gene (CAT) under the transcriptional control of the cytomegalovirus immediate early promoter (CMV-IE) and inserting the coding region of CD83 downstream of the CAT gene followed by a poly(A) site. The plasmid pBC12/HIV/CAT (Berger, J. et al. (1988) Gene 66: 1-10) was digested with HindIII and BamHI in order to isolate a DNA fragment containing the CAT gene. The plasmid pBC12/CMV/P-Gal/SD-SA was also digested with HindIII and BamHI and the HindIII and BamHI digested CAT gene was inserted into HindIII and BamHI digested plasmid pBC12/β-Gal/SD-SA such that the β-Gal of pBC12/CMV/P-Gal/SD-SA was replaced by the CAT gene. This CAT containing vector was then digested with BamHI and XmaI in order to remove the SD-SA region and various sub-fragments of the CD83 gene were cloned into this BamHI and XmaI digested vector (see FIG. 11. A CAT containing vector without CD 83 sequences was also constructed as a negative control for some experiments by filling in the 5′ overlapping ends created by digestion with BamHI and XmaI with Klenow fragment and ligating these ends together.

A series of CD83 sub-fragments without start and stop codons were obtained using the following primers or oligonucleotides in a standard PCR reaction as described above and were ligated into the BamHI and XmaI sites in the 3′ non-translated region of the CAT gene of the above pBC12 vector:

Sub-fragment CD83 nt412-nt615 (=aa138-aa205):

5′ primer; introduces BamHI site:

5′ -ATTTAAGGATCCTTTAAGAAATACAGAGCGGAGATTGTCCTG-3′ (SEQ ID NO:15) and

3′ primer; introduces XmaI site:
5′-ATTTAACCCGGGTACCAGTTCTGTCTTGTGAGGAGTCACTAG-3′
(SEQ ID NO:16);
Sub-fragment CD83 nt412-nt615 (= aa138-aa205)
antisense
5′ primer; introduces XmaI site:
5′-ATTTAACCCGGGTTTAAGAAATACAGAGCGGAGATTGTCCTG-3′
(SEQ ID NO:17) and
3′ primer; introduces BamHI site:
5′-ATTTAAGGATCCTACCAGTTCTGTCTTGTGAGGAGTCACTAG-3′
(SEQ ID NO:18);
Sub-fragment CD83 nt412-nt465 (aa138-aa155)
Oligos for direct annealing:
5′ primer; introduces BamHI site:
5′-GATCCTTTAAGAAATACAGAGCGGAGATTGTCCTGCTGCTGG
CTC . . . TGGTTATTTTCTACC-3′ (SEQ ID NO:19) and
3′ primer; introduces XmaI site:
5′-GAAATTCTTTATGTCTCGCCTCTAACAGGACGACGACCGAGA
CCA . . . ATAAAAG-3′ (SEQ ID NO:20);
Sub-fragment CD83 nt466-nt615 (= aa156-aa205)
5′ primer; introduces BamHI site:
5′-ATTTAAGGATCCTTAACACTCATCATTTTCACTTGTAAGTTTGC-3′
(SEQ ID NO:21) and
3′ primer; introduces XmaI site:
5′-ATTTAACCCGGGTACCAGTTCTGTCTTGTGAGGAGTCACTAG-3′
(SEQ ID NO:16)

These vectors were then transiently transfected into COS cells (ATCC number:CRL-1650) via DEAE dextran transfection. Briefly, 6-well culture dishes (Corning, Corning, USA) were treated with 0.1% gelatin for 20 min. at room temperature and then washed twice with PBS. 2.5×10⁵ COS cells were seeded in 4 ml culture medium (DMEM (Gibco, Karlsruhe, Germany) with 10% fetal calf serum, 1% non-essential amino acids, 1% glutamine and 1% penicillin/streptomycin) and cultured overnight. All DNAs to be transfected were brought to a final concentration of 0.025 [μg/ml. In a sterile Eppendorf tube, 25 μl of the DNA solution containing the CD83 constructs were mixed with pBC12/CMV vector to obtain a final DNA concentration of 2.5-3.0 [g/ml. 225 μl of a DEAE dextran solution (62.5 μl of DEAE dextran stock solution (20 mg/ml) in 2.24 ml PBS) was added to the 25μl DNA solution and this was carefully added directly to the COS cells after the culture medium was removed and the cells were washed twice with 37° C. PBS. The transfection reactions were allowed to progress for 30 min in an incubator (37° C., 5% CO₂, 80% humidity). Then, 2.5 ml of FC medium (20 ml of the DMEM medium described above with 400 μl fungizone (2%) and 20 μl chloroquine (100 mM) were added to each well and the cells were incubated for a further 2.5 hours at 37° C., 5% CO₂, 80% humidity. The DNA/FC medium mixture was then aspirated and each well received 1 ml of shock medium consisting of 9 ml the DMEM culture medium described above and 1 ml DMSO. After 2.5 min. incubation at room temperature, the shock medium was aspirated and replaced by culture medium. Incubation continued for 48-72 hours at 37° C., 5% CO₂, 80% humidity.

In order to exclude erroneous results generated by different transfection efficiencies, each transfection was performed with the same amount of an internal control vector constructed by digesting the vector pBC12/CMV/β-Gal/SD-SA with BamHI and XmaI, filling in the 5′ overlapping ends with Klenow fragment (Roche Diagnostics, Mannheim, Germany) and religating the vector.

Lysis of the COS cells transfected in this manner occurred with the cell lysis buffer of the CAT ELISA kit of Roche Diagnostics, Mannheim, Germany, in accordance with the manufacturer's instructions.

Evaluation of the assay was performed by CAT ELISA and β-Gal assay.

For the CAT ELISA, 200 μl of transfected cell extract was used and the measurement of the color reaction occurred after 30 min. in an ELISA microplate reader at the wave lengths of 405 nm and 490 nm (reference).

For the β-Gal assay, 25 μl of transfected cell extract was added to 200 μl of Working Z buffer (10 ml Z buffer (16,1 gr Na₂HPO₄ * 7 H₂O, 5,5 gr NaH₂PO₄ * H₂O, 0.75 gr KCl, 0,25 gr MgSO₄ * 7 H₂O in 1 liter water, to pH 7.0) to which 8 μl of 10% SDS and 28 μl β-mercaptoethanol are added directly before use) in a 96 well microtiter plate. Then, 25 ml CPRG (chlorophenol red-β-D-galactopyranoside (Roche Diagnostics, Mannheim, Germany); 15 mg dissolved in 1 ml H₂O) was added and after about 10 min., measurement occurred using an ELISA reader at a wave length of 560 nm.

In the following experiments, the CAT activity is given with respect to the β-Gal activity. The background activity was measured by transformation of the cells with a plasmid without a CAT gene and was subtracted from the measured values to obtain a normalized CAT activity. Unless otherwise noted, the experiments were carried out at least 5 times for statistical evaluation.

### Example 8

In order to ascertain whether a difference in the measured CAT activity exists between a reporter construct with and without a CD83 insert, transfection experiments were first carried out with the CAT containing vector without CD83 sequences (see Example 7) and the CAT containing vector with the CD83 insert from nucleotides 412 to 615 of SEQ ID NO:1.

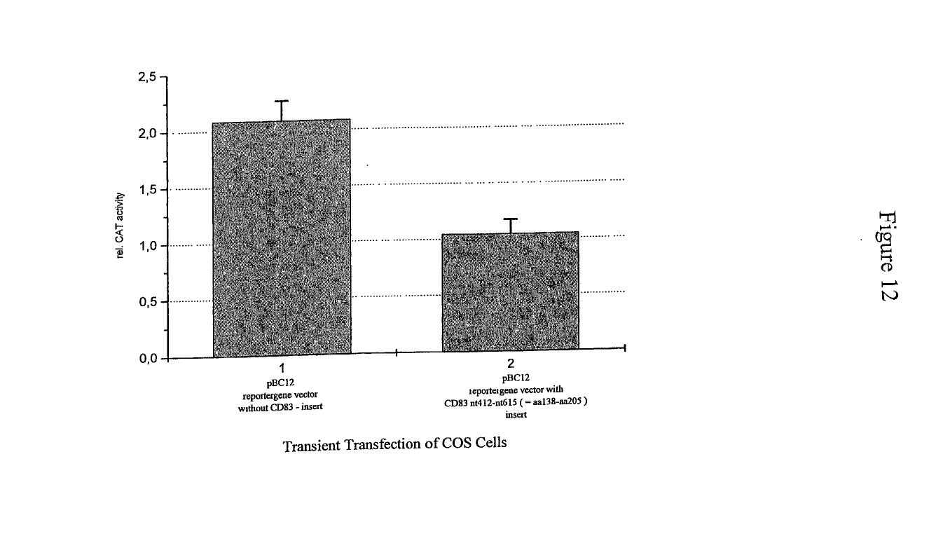

An approximate two-fold decrease in the CAT activity from the vector with the CD83 insert from nucleotides 412 to 615 of SEQ ID NO:1 was observed in comparison to the CAT containing vector without CD83 sequences (see FIG. 12).

In order to determine a possible effect of HuR on the observed CAT activity of a CAT containing vector with a CD83 insert, COS cells were co-transfected with the same amounts of the plasmid pcDNA-ELAV (HuR) and the vector with the CD83 insert from nucleotides 412 to 615 of SEQ ID NO:1. The vector pcDNA3-ELAV (HuR) comprises the coding region of HuR as given in SEQ ID NO:5 cloned into the HindIII and XhoI sites of pcDNA3 under control of the CVM-IE promoter.

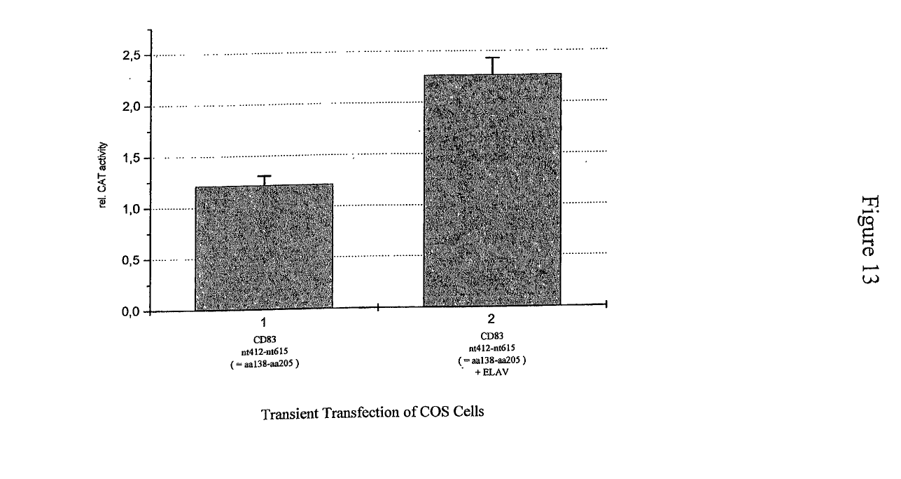

As can be seen from FIG. 13, an approximate two-fold increase in CAT activity was measured with co-transfection of the ELAV (HuR) vector as compared to transfection with the vector with the CD83 insert from nucleotides 412 to 615 of SEQ ID NO:1 alone.

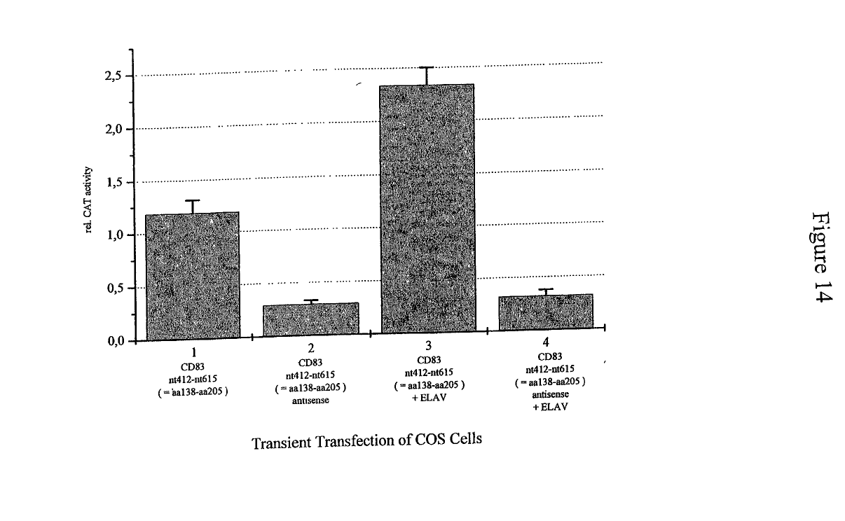

In addition to the above, in experiments using a CAT containing vector with the CD83 insert from nucleotides 412 to 615 of SEQ ID NO:1 in the sense or antisense orientation, it could be demonstrated that a) the CAT containing vector with the sense orientation of the CD83 sequence has an approximately 5-fold greater CAT activity than the antisense vector in COS cells which were not transfected with the pcDNA-ELAV (HuR) plasmid and b) the CAT activity of the vector with the CD83 insert from nucleotides 412 to 615 of SEQ ID NO:1 in the sense orientation is increased approximately two-fold by co-transfection with the pcDNA-ELAV (HuR) plasmid as compared to the CAT activity of the antisense vector (see FIG. 14).

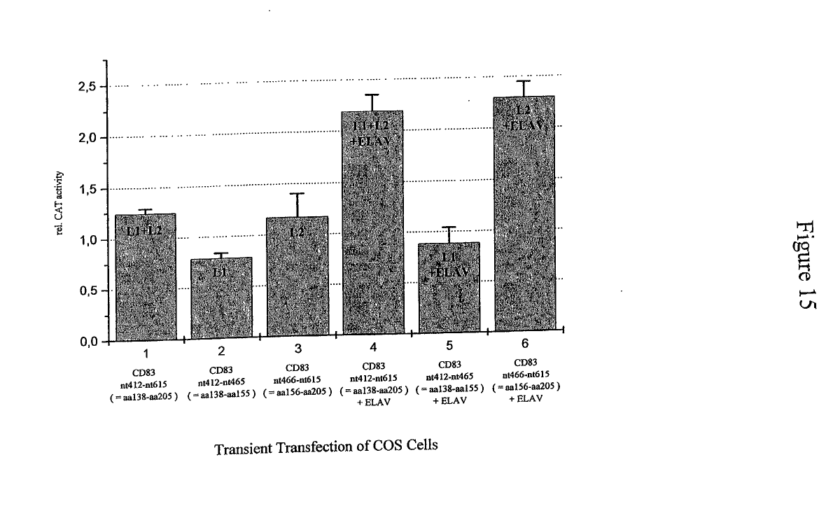

Moreover, in experiments in which COS cells were transiently transfected using a CAT containing vector with the CD83 insert from nucleotides 412 to 615 of SEQ ID NO:1, a CAT containing vector with the CD83 insert from nucleotides 412 to 465 of SEQ ID NO:1 (stem-loop-1) or a CAT containing vector with the CD83 insert from nucleotides 466 to 615 of SEQ ID NO:1 (stem-loop-2), with or without the pcDNA-ELAV (HuR) plasmid, it could be demonstrated that the CAT containing vectors with stem-loop-2 (i.e. comprising nucleotides 412 to 615 of SEQ ID NO:1 or nucleotides 466 to 615 of SEQ ID NO:1) lead to an approximately two-fold greater CAT activity when COS cells were co-transfected with the pcDNA-ELAV (HuR) plasmid as compared to the CAT activity of COS cells that were transfected with CAT containing vectors with stem-loop-2 alone (see FIG. 15).

No HuR-dependent effect could be shown for the CAT containing vector with the CD83 insert from nucleotides 412 to 465 of SEQ ID NO:1 (stem-loop-1).

Summarizing the above results, it was demonstrated that the HuR protein is capable of specifically binding to an RNA molecule that comprises at least a portion of the nucleotide sequence coding for a region of the CD83 protein from nucleotides 466 to 615 of SEQ ID NO:1. Furthermore, it was demonstrated in an in vitro transient expression system that the over-expression of HuR leads to an increase in the amount of protein expressed from a mRNA molecule comprises at least a portion of the nucleotide sequence coding for a region of the CD83 protein from nucleotides 466 to 615 of SEQ ID NO:1.

### Example 10

A DNA sequence containing nucleotides 466 to 615 of SEQ ID NO:1 encoding the amino acids 156 to 205 of CD83 was used as input data in the MFOLD program to calculate the secondary structure of the corresponding RNA molecule. A folding temperature of 37° C. was used. The most probable secondary structure based on this calculation, i.e. the secondary structure having the most negative energy in kcal/mol is presented in FIG. 16 (−29.7 kcal/mol) and the second most probable in FIG. 17 (−28.4 kcal/mol).

Each of these secondary structures has a domain (stem-loop-2) comprising a single 3-pronged stem-loop structure and containing the CD83 RNA sub-fragment from nucleotide 466 to nucleotide 615 of SEQ ID NO:1.

### Example 11

Specific protein-RNA interactions were detected by Surface Plasmon Resonance using the BIACore X optical biosensor (Pharmacia Biosensor AB, Upsala, Sweden).

The RNA used contained nucleotides 466 to 615 of SEQ ID NO:1 having a molecular mass of 48,350 gr/mol.

The GST-fusion proteins of interest (GST alone or GST-ELAV (HuR)) were immobilized onto the surface of a CM5 Chip (Pharmacia Biosensor AB, Upsala, Sweden) by using the BIACore GST Capture Kit (Pharmacia Biosensor AB, Upsala, Sweden), according to the manufacturers instructions, until a change of at least 1,000 resonance units (RU) was detectable.

1 μg of in vitro transcribed RNA (using the T7/SP6 Riboprobe Kit, Promega, Mannheim, Germany) was diluted in a total volume of 80 μl HBS-EP buffer (10 mM HEPES, 150 mM NaCl, 3 mM EDTA, 0.005% (v/v) polysorbate 20) and was allowed to flow over the chip at a flow rate of 50μl/minute. The standard running buffer was HBS-EP buffer. The sensor surface was regenerated between assays by injecting 30 μl of 0.5 M NaCl to remove bound analyte.

Determination of K_{D} values and calculation of binding specifity were performed using BIAEvaluation Software 3.1 (Pharmacia Biosensor AB, Upsala, Sweden).

The K_{D} value of the binding of HuR to a portion of the CD83 from nucleotides 466 to 615 of SEQ ID NO:1 was determined to be 7.0-8.0×10⁻⁶ M. Thus, it could be shown that binding of HuR to this portion of the CD83 mRNA occurs with high affinity and is highly specific.

1. A compound that specifically blocks the binding between a member of the HuR family of proteins and a mRNA encoding a member of the CD83 family of proteins and that reduces expression of a member of the CD83 family of proteins in a cell.

2. A compound according to claim 1, characterized in that said compound comprises a nucleic acid.

3. A compound according to claim 1 or 2, characterized in that the compound comprises DNA.

4. A compound according to claim 1 or 2, characterized in that the compound comprises RNA.

5. A compound according to any of the preceding claims, characterized in that the compound contains at least a portion of the coding region of a member of the CD83 family of proteins or a derivative thereof.

6. A compound according to any of the preceding claims, characterized in that the compound contains from nucleotide 466 to 618 of the sequence in SEQ ID NO:1 or a derivative thereof.

7. A compound according to any of the preceding claims, characterized in that the compound contains from nucleotide 466 to 615 of the sequence in SEQ ID NO:1 or a derivative thereof.

8. A compound according to any of the preceding claims, characterized in that the compound contains a nucleic acid having a secondary structure comprising a 3-pronged stem-loop structure with an energy of −28.4 kcal/mol or less, preferably −29.7 kcal/mol or less.

9. A compound according to any of the preceding claims, characterized in that the compound is a nucleic acid that comprises regulatory sequences that lead to the transcription of an RNA molecule from said nucleic acid in a cell.

10. A compound according to any of the preceding claims, characterized in that the compound is a nucleic acid that does not contain regulatory sequences that lead to the translation of a polypeptide or protein from said nucleic acid in a cell.

11. A compound according to claim 1, characterized in that said compound comprises a protein.

12. A compound according to claim 11, characterized in that said protein is a derivative of members of the ELAV superfamily of proteins.

13. A compound according to claim 12, characterized in that said protein is selected from the group of proteins consisting of ELAV, HuR, HuB, HuC, HuD, HuDpro, HuDmex, Hel-N2 and HuC isoforms, Rel-N1 and naturally occurring homologues of these proteins.

14. A compound according to claim 11, characterized in that said protein is a derivative of a protein ligand to HuR.

15. A compound according to claim 14, characterized in that said protein is selected from the group of proteins consisting of SETα, SETβ, pp32 and APRIL as well as naturally occurring homologues of these proteins.

16. A pharmaceutical composition comprising a compound according to any of claims 1 to 15.

17. The use of a compound according to any of claims 1 to 15 for the production of a pharmaceutical composition for treating or preventing disease involving the direct or indirect participation of DC.

18. The use according to claim 17, characterized in that said disease is selected from the group consisting of diseases involving the growth, differentiation and/or activation of cytotoxic T cells and helper T cells, the differentiation of helper T cells into Th1 cells or Th2 cells, the growth, stimulation and/or differentiation of B cells.

19. The use according to claim 17, characterized in that said disease is selected from the group consisting of allergies, asthma autoimmune syndromes such as myasthemia gravis, multiple sclerosis and systemic lupus erythematosis, skin diseases such as psoriasis, rheumatoid arthritis and AIDS.

20. The use of a compound a compound according to any of claims 1 to 15 for the production of a pharmaceutical composition for treating or preventing rejection of a tissue or organ transplant.

21. An expression vector comprising a nucleic acid sequence encoding a ribonucleic acid according to any of claims 4 to 10.

22. A host cell transformed with the expression vector according to claim 21.

23. The use of an expression vector encoding an ribonucleic acid according to any of claims 4 to 10 for the production of a medicament for the treatment and prevention of disorders, diseases and syndromes involving the direct or indirect participation of DC by regulating an immune response.

24. An expression vector comprising a nucleic acid sequence encoding a protein according to any of claims 11 to 15.

25. A host cell transformed with the expression vector according to claim 24.

26. The use of an expression vector encoding a protein according to any of claims 11 to 15 for the production of a medicament for the treatment and prevention of disorders, diseases and syndromes involving the direct or indirect participation of DC by regulating an immune response.

27. Method for screening and/or identifying compounds that block the binding between a member of the HuR family of proteins and a mRNA encoding a member of the CD83 family of proteins comprising the steps of incubating one or more compounds in a reaction comprising:
(a) a nucleic acid molecule that contains at least a portion of the coding region of a member of the CD83 family of proteins or a derivative thereof and
(b) a member of the ELAV superfamily of proteins or derivative thereof
under conditions sufficient to allow the components to interact and determining whether the compound blocks the binding between the nucleic acid molecule and the member of the ELAV superfamily of proteins.

28. Method according to claim 27, characterized in that the member of the CD83 family of proteins is CD83.

29. Method according to claim 27 or 28, characterized in that the member of the ELAV superfamily of proteins is selected from the group consisting of ELAV, HuR, HuB, HuC, HuD, HuDpro, HuDmex, Hel-N2 and HuC isoforms, Rel-N1 and naturally occurring homologues of these proteins.

30. Method according to any of claims 27 to 29, characterized in that said method is carried out in the form of an assay selected from the group RNA gel shift assay, filter binding assay, Biacore interaction analysis, Scintilation Proximity Assay, RNAse protection assay, cell-based RNA binding assay, yeast 3-hybrid assays and reporter gene assay.

31. Use of a compound that blocks the binding between a member of the HuR family of proteins and a mRNA encoding a member of the CD83 family of proteins when incubated in a reaction comprising:
(a) a nucleic acid molecule that contains at least a portion of the coding region of a member of the CD83 family of proteins or a derivative thereof and
(b) a member of the ELAV superfamily of proteins or derivative thereof
under conditions sufficient to allow the components to interact, for the production of a pharmaceutical composition for treating or preventing disease involving the direct or indirect participation of DC.
